SEQUENCE LISTING (31 sequences)

SEQ ID NO: 1 - protein, 15 aa, Artificial sequence
  Citrullinated peptide
  X (1)..(1): X = citrullyl residue or arginyl residue
  X (13)..(13): X = citrullyl residue or arginyl residue
  X (15)..(15): X = citrullyl residue or arginyl residue
XPAPPPISGGGYXAX

SEQ ID NO: 2 - protein, 15 aa, Artificial sequence
  Citrullinated peptide
  X (3)..(3): X = citrullyl residue or arginyl residue
  X (7)..(7): X = citrullyl residue or arginyl residue
GPXVVEXHQSACKDS

SEQ ID NO: 3 - protein, 15 aa, Artificial sequence
  Citrullinated peptide
  X (10)..(10): X = citrullyl residue or arginyl residue
  X (12)..(12): X = citrullyl residue or arginyl residue
SGIGTLDGFXHXHPD

SEQ ID NO: 4 - protein, 15 aa, Artificial sequence
  Citrullinated peptide
  X (8)..(8): X = citrullyl residue or arginyl residue
  X (11)..(11): X = citrullyl residue or arginyl residue
VDIDIKIXSCXGSCS

SEQ ID NO: 5 - protein, 15 aa, Artificial sequence
  Citrullinated peptide
  X (3)..(3): X = citrullyl residue
  X (7)..(7): X = citrullyl residue
GPXVVEXHQSACKDS

SEQ ID NO: 6 - protein, 15 aa, Artificial sequence
  Citrullinated peptide
  X (8)..(8): X = citrullyl residue
  X (11)..(11): X = citrullyl residue
VDIDIKIXSCXGSCS

SEQ ID NO: 7 - protein, 15 aa, Artificial sequence
  Citrullinated peptide
  x (4)..(4): X = citrullyl residue
  x (8)..(8): X = citrullyl residue
SCSXALAXEVDLKDY

SEQ ID NO: 8 - protein, 15 aa, Artificial sequence
  Citrullinated peptide
  X (13)..(13): X = citrullyl residue
PEWKALTDMPQMXME

SEQ ID NO: 9 - protein, 15 aa, Artificial sequence
  Citrullinated peptide
  X (5)..(5): X = citrullyl residue
  X (13)..(13): X = citrullyl residue
MELEXPGGNEITXGG

SEQ ID NO: 10 - protein, 15 aa, Artificial sequence
  Citrullinated peptide
  X (2)..(2): X = citrullyl residue
EXGSAGHWTSESSVS

SEQ ID NO: 11 - protein, 15 aa, Artificial sequence
  Citrullinated peptide
  X (9)..(9): X = citrullyl residue
DSPGSGNAXPNNPDW

SEQ ID NO: 12 - protein, 15 aa, Artificial sequence
  Citrullinated peptide
  X (1)..(1): X = citrullyl residue or arginyl residue
  X (7)..(7): X = citrullyl residue or arginyl residue
  X (10)..(10): X = citrullyl residue or arginyl residue
XGHAKSXPVXGIHTS

SEQ ID NO: 13 - protein, 15 aa, Artificial sequence
  Citrullinated peptide
  X (15)..(15): X = citrullyl residue
GTFEEVSGNVSPGTX

SEQ ID NO: 14 - protein, 15 aa, Artificial sequence
  Citrullinated peptide
  X (10)..(10): X = citrullyl residue
  X (12)..(12): X = citrullyl residue
SGIGTLDGFXHXHPD

SEQ ID NO: 15 - protein, 15 aa, Artificial sequence
  Citrullinated peptide
  X (2)..(2): X = citrullyl residue
SXGSESGIFTNTKES

SEQ ID NO: 16 - protein, 15 aa, Artificial sequence
  Citrullinated peptide
  X (13)..(13): X = citrullyl residue
SSHHPGIAEFPSXGK

SEQ ID NO: 17 - protein, 15 aa, Artificial sequence
  Citrullinated peptide
  X (4)..(4): X = citrullyl residue
SYNXGDSTFESKSYK

SEQ ID NO: 18 - protein, 15 aa, Artificial sequence
  Citrullinated peptide
  X (1)..(1): X = citrullyl residue
  X (7)..(7): X = citrullyl residue
  X (10)..(10): X = citrullyl residue
XGHAKSXPVXGIHTS

SEQ ID NO: 19 - protein, 15 aa, Artificial sequence
  Citrullinated peptide
  X (1)..(1): X = citrullyl residue
  X (13)..(13): X = citrullyl residue
  X (15)..(15): X = citrullyl residue
XPAPPPISGGGYXAX

SEQ ID NO: 20 - protein, 15 aa, Artificial sequence
  Citrullinated peptide
  X = citrullyl residue (15)..(15): X = citrullyl residue
QKLESDVSAQMEYCX

SEQ ID NO: 21 - protein, 15 aa, Artificial sequence
  Citrullinated peptide
  X (5)..(5): X = citrullyl residue
  X (14)..(14): X = citrullyl residue
VIQNXQDGSVDFGXK

SEQ ID NO: 22 - protein, 15 aa, Artificial sequence
  Citrullinated peptide
  X (2)..(2): X = citrullyl residue
PXKQCSKEDGGGWWY

SEQ ID NO: 23 - protein, 15 aa, Artificial sequence
  Citrullinated peptide
  X (4)..(4): X = citrullyl residue
  X (13)..(13): X = citrullyl residue
WYNXCHAANPNGXYY

SEQ ID NO: 24 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 234 aa, Artificial sequence
  Fc Fragment + Qtag
LLQGARSDATHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 241 aa, Artificial sequence
  Fc Fragment + Qtag
AHGHGHGLLQGARSDATHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 600 aa, Homo sapiens
GPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTS

SEQ ID NO: 28 - protein, 447 aa, Homo sapiens
GHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 29 - protein, 4 aa, Artificial sequence
  Qtag
LLQG

SEQ ID NO: 30 - protein, 635 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTS

SEQ ID NO: 31 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

(19) United States
(12) Patent Application Publication
(10) Pub. No.: US 2024/0294658 A1
(43) Pub. Date: Sep. 5, 2024

(54) HYBRID MOLECULE COMPRISING A FIBRIN-DERIVED CITRULLINATED PEPTIDE AND AN ANTIBODY OR ANTIBODY FRAGMENT WHICH BINDS TO CD38 AND/OR CD138, AND USES THEREOF

(71) Applicants: UNIVERSITE TOULOUSE III - PAUL SABATIER, Toulouse (FR); INSERM (INSTITUT NATIONAL DE LA SANTÉ ET DE LA RECHERCHE MÉDICALE), Paris (FR); CENTRE HOSPITALIER UNIVERSITAIRE DE TOULOUSE, TOULOUSE (FR); CENTRE HOSPITALIER UNIVERSITAIRE DE MONTPELLIER, Montpellier (FR); UNIVERSITE DE MONTPELLIER, Montpellier (FR); ARTHRITIS RECHERCHE & DEVELOPPEMENT, NEUILLY SUR SIENE (FR)

(72) Inventors: Pauline VACHIN, MONTPELLIER (FR); Pascale LOUIS-PLENCE, SAINT MATHIEU DE TREVIERS (FR); Christian JORGENSEN, SAINT-CLEMENT DE RIVIERE (FR); Camille ESQUERRE, AUZIELLE (FR); Cyril CLAVEL, SAINT FOY DE PEYROLIERES (FR); Guy Bruno René SERRE, TOULOUSE (FR); Eve COMBES, VILLEVEYRAC (FR); Bruno ROBERT, MONTPELLIER (FR); Pierre MARTINEAU, MONTPELLIER (FR)

(21) Appl. No.: 18/551,146

(22) PCT Filed: Mar. 18, 2022
(86) PCT No.: PCT/FR2022/050505

§ 371 Date: Sep. 18, 2023

(30) Foreign Application Priority Data
Mar. 19, 2021 (FR) FR2102804

(51) Int. Cl. C07K 16/28; A61K 38/00; C07K 14/745

(52) U.S. Cl. CPC C07K 16/2896; C07K 14/745; A61K 38/00; C07K 2317/54; C07K 2317/55; C07K 2317/732; C07K 2319/30

(57) ABSTRACT

The invention relates to a hybrid molecule comprising a fibrin-derived citrullinated peptide and an antibody or antibody fragment which binds to CD38 and/or CD138, the uses of such a hybrid molecule and its method of production.

### SEQUENCE LISTING SUBMISSION VIA PATENT CENTER

The contents of the electronic sequence listing (SequenceListing.txt; Size: 34,307 bytes; and Date of Creation: Mar. 26, 2024) is herein incorporated by reference in its entirety.

The present invention relates to a hybrid molecule comprising a fibrin-derived citrullinated peptide and an antibody or antibody fragment which binds to CD38 and/or CD138, the uses of such a hybrid molecule and its method of production.

### CONTEXT OF THE INVENTION

Rheumatoid arthritis is the most common type of inflammatory rheumatism or arthritis, but also the most common autoimmune disease. The disease is characterised by chronic inflammation of the synovial joints, resulting in irreversible joint destruction.

The presence of class G autoantibodies directed against citrullinated proteins, known as anti-citrullinated protein autoantibodies (ACPAs), is highly specific for rheumatoid arthritis. Serum from patients that contains ACPAs, the lymphocyte cells that express them on their membrane and the patients themselves are all said to be “ACPA-positive”. Several studies have demonstrated that these ACPAs are at the heart of the autoimmune reactions specific to rheumatoid disease and thus represent a therapeutic target of choice.

The antigenic targets of ACPAs have been characterised. They are directed, in particular, specifically against deiminated, or citrullinated, forms of the α and β polypeptide chains of fibrin, a protein abundant in inflamed synovial tissue. This citrullination corresponds to the enzymatic deimination of the arginyl residues of a protein by the action of peptidyl-arginine deiminases (PAD).

More specifically, the immunodominant epitopes recognised by ACPAs on the α and β polypeptide chains of fibrin have been characterised and published, in particular in applications PCT/FR00/01857 or PCT/FR2007/000758. The five citrullinated peptides carrying immunodominant epitopes are more particularly the peptides named α36-50Cit (as shown in SEQ ID NO: 5), α171-185Cit (as shown in SEQ ID NO: 6), α501-515Cit (as shown in SEQ ID NO: 14), α621-635Cit (as shown in SEQ ID NO: 18) and β60-74Cit (as shown in SEQ ID NO: 19). Sera from ACPA-positive patients recognise one or more of these five peptides.

Secreted into the rheumatoid synovial tissue by local plasma cells, ACPAs are found there in high concentrations. They remain close to their main target, citrullinated fibrin, which is also abundant there in the form of interstitial deposits. The binding of ACPAs to these deposits and the resulting formation of immobilised immune complexes that in turn bind to rheumatoid factors, another autoantibody associated with rheumatoid arthritis and also secreted by local plasma cells triggers a cascade of pro-inflammatory events.

The stimulation of macrophage cells by these immune macro-complexes, essentially via their membrane Fc-gamma receptors, leads them to secrete pro-inflammatory cytokines and in particular TNF-alpha, which has been identified as the main cytokine responsible for rheumatoid inflammation.

To date, there is no curative treatment for rheumatoid arthritis. Treatments are aimed only at treating and/or preventing the occurrence of flare-ups.

An object of the present invention is thus to provide a treatment for rheumatoid arthritis.

ACPAs are oligoclonal and therefore secreted by only a few clonal plasma cells, themselves resulting from the differentiation of a few clonal B lymphocytes.

In the case of rheumatoid arthritis, clonal B lymphocytes express on their membranes immunoglobulins carrying ACPA specificity (these are ACPA-positive B lymphocytes), whereas plasma cells resulting from the differentiation of ACPA-positive clonal B lymphocytes (these are ACPA-positive plasma cells) secrete these ACPAs in abundance into their microenvironment.

The present invention is based on the results of the inventors showing that it is possible to target plasma cells secreting ACPAs in abundance in their microenvironment and to eliminate them using a hybrid molecule comprising (i) a fibrin-derived peptide having at least one citrullyl residue and (ii) an antibody or antibody fragment which binds to CD38 and/or CD138. These hybrid molecules will target plasma cells (using the antibody or antibody fragment which binds to CD38 and/or CD138, which are molecular markers expressed on the surface of said plasma cells) and, thanks to the fibrin-derived peptide having at least one citrullyl residue, the hybrid molecule will be recognised specifically by the ACPAs (the peptide being the target of these ACPAs). After bridging (binding of ACPAs to the hybrid molecule, itself bound to plasma cells), the plasma cells will be lysed by apoptosis, by phagocytosis in the presence of macrophages, by ADCC (antibody-dependent cell-mediated cytotoxicity) in the presence of NK cells, or by complement activation.

By targeting the plasma cells that secrete ACPAs, the hybrid molecules of the invention thus aim to cause the disappearance of the ACPAs from the patients' bodies and of the source of these ACPAs to induce a remission of the disease.

The targeting of plasma cells and of the antibodies released by said plasma cells using a hybrid has already been described, for example in application EP2892926. However, it discloses no specific construct or result relating to rheumatoid arthritis.

### DISCLOSURE OF THE INVENTION

### Hybrid Molecule According to the Invention

In a first aspect, the invention relates to a hybrid molecule comprising at least one fibrin-derived peptide having at least one citrullyl residue, said peptide being covalently bound to at least one antibody or to at least one antibody fragment, said antibody or fragment being capable of binding to CD38 and/or CD138, one or more spacers being optionally present between said peptide and said antibody or said fragment. The diagram of such a construct is presented in FIG. 2.

According to the invention, a “hybrid molecule” means a molecule having at least two components of different nature, in this case said peptide and an antibody or antibody fragment capable of binding to CD38 and/or CD138.

According to the invention, an “antibody” means an immunoglobulin. Immunoglobulins consist of an assembly of two dimers, each consisting of a heavy chain and a light chain. Each of the heavy and light chains consist of a constant region and a variable region. More specifically, each light chain consists of a variable region (VL) and a constant region (CL). Each heavy chain consists of a variable region (VH) and a constant region consisting of three or four constant domains CH1, CH2, CH3 and optionally CH4. Antibody means IgM, IgD, IgG, IgA and IgE. Preferably, the antibody is an IgG, more particularly IgG1. An antibody according to the invention also means a bispecific antibody, which has an affinity for two different antigens. The antibody used in the hybrid molecule according to the invention is preferably a monoclonal antibody.

According to the invention, an “antibody fragment” means a portion of antibody that carries the antigen-binding site. Such antibody fragments are, for example, Fab, F(ab′)2, Fv, dsFv, scFv, single-chain fragments such as isolated VH or VL fragments, camelid VHHs, cartilaginous fish VNARs, or even multispecific antibodies consisting of different fragments, such as bispecific antibodies, or “nanobodies”, “diabodies”, “triabodies”, “tetrabodies”, or scFvs in tandem. These fragments are well known to those skilled in the art. Further information concerning these fragments and constructs is described, for example, in international application WO2017137579, in Bird et al., 1988 Science 242:423-426; and in Huston et al., 1988 Proc. Natl. Acad. Sci. 85:5879-5883, or in Nelson, mAbs 2:1, 77-83; January/February 2010.

Typically, a “Fab” is an antibody fragment that comprises about half of the N-terminal side of the heavy chain and the entire light chain of an antibody, linked by a disulfide bridge. The Fab can be obtained in particular from an IgG by treatment with a protease, papain. An “F(ab′)2” thus designates two Fab fragments as defined above, linked together by a disulfide bridge. The F(ab′)2 can be obtained in particular from an IgG by treatment with a protease, pepsin.

Typically, an “Fv” corresponds to two non-covalently bound VL and VH domains and a “dsFv” corresponds to the association of a VH and VL linked by a disulfide bridge.

Typically, an “scFv” (single chain Fv) is a VH:VL polypeptide synthesised using the genes encoding the VL and VH domains and a sequence encoding a peptide intended to link these domains. The scFvs can be associated with each other and form, for example, diabodies (2 scFvs), triabodies (3 scFvs) or tetrabodies (4 scFvs). In such constructs, the scFvs are generally linked together by one or more peptide bonds. The antigenic specificity of each scFv involved in such multimeric constructs may be the same or different.

According to a preferred embodiment, in said hybrid molecule, said fragment is selected from fragments scFv, Fv, Fab or F(ab′)2, more particularly Fab or F(ab′)2.

According to the invention, “CD38” and “CD138” refer to markers expressed on the surface of plasma cells. CD138 is specific for plasma cells. CD38 is not specific for plasma cells, but plasma cells are nevertheless among the cells that most strongly express this marker. Daratumumab is an example of a therapeutic antibody that targets CD38. Preferably, said antibody or said fragment is directed against CD38.

According to another embodiment, in said hybrid molecule, said antibody or said fragment is bispecific, and is directed against: CD38 and another plasma cell target or CD138 and another plasma cell target or CD38 and CD138. More particularly, according to one embodiment, in said hybrid molecule, said antibody or F(ab′)2 fragment is a bispecific antibody or F(ab′)2 fragment, directed against:
CD38 and another plasma cell target, or
CD138 and another plasma cell target, or
CD38 and CD138.

According to the invention, “another plasma cell target” means any marker expressed on the surface of the plasma cells.

According to the invention, the expression “covalently bound” means a covalent bond, that is, a chemical bond in which two atoms share two electrons. Said covalent bond can be polar or non-polar.

According to the invention, a “spacer” is a coupling agent that enables covalent bonding of said antibody or antibody fragment to said fibrin-derived peptide having at least one citrullyl residue, while separating said antibody or antibody fragment from said peptide (thus reducing possible steric hindrance). The spacer can be any molecule, and in particular a peptide. Preferably, the spacer does not modify the physical and chemical properties of the hybrid molecule.

The presence of at least one spacer is advantageous: it facilitates the independent accessibility of the two partners of the hybrid molecule (the antibody or antibody fragment is more easily accessible for binding to the plasma cells, just as said peptide is more easily accessible for binding to ACPA-positive B cells), and/or it stabilises the hybrid molecule, and/or increases the solubility of the hybrid molecule.

According to one embodiment, the hybrid molecule according to the invention may comprise one or more spacers. Preferably, the hybrid molecule comprises one or two spacers. According to one embodiment, when at least one spacer is present in said hybrid molecule of the invention, said antibody or antibody fragment is covalently bound to a spacer, said spacer being itself covalently bound to said peptide. According to another embodiment, when at least two spacers are present in said hybrid molecule of the invention, said antibody or antibody fragment is covalently bound to a first spacer, said first spacer being itself covalently bound to a second spacer and the second spacer being itself covalently bound to said peptide. The bond between the antibody or antibody fragment and the peptide can therefore be direct, or even indirect in the presence of spacers.

According to one embodiment, the hybrid molecule according to the invention may comprise at least one fibrin-derived peptide having at least one citrullyl residue. This means that the antibody or antibody fragment can be linked to one or two peptides. This is because the antibody or antibody fragment comprises two monomers, and the antibody or antibody fragment can thus be covalently bound to a peptide on only one of the two monomers, or the antibody or antibody fragment can be covalently bound to a peptide on each monomer (even three or four in the case of a bispecific antibody or fragment). Preferably, when two peptides are bound to the antibody or antibody fragment, the two peptides are identical, for example two peptides of SEQ ID NO: 18 or SEQ ID NO: 19.

According to one embodiment, said spacer is a polymer containing one or more repeat units containing the ether group. According to one particular embodiment, said spacer is polyethylene glycol of formula PEGn, in which n represents an integer between 1 and 100, preferably between 1 and 10, and in particular 1, 2, 3, 4 or 8. According to the invention, said polyethylene glycol can be functionalised, for example with an amine group (PEGn-amine such as PEG-NH₂). According to the invention “an integer between 1 and 100” represents all the integer values between 1 and 100, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100.

According to the invention, the expression “fibrin-derived peptide having at least one citrullyl residue” means a molecule of fibrin or fibrinogen in which at least one arginyl residue has been substituted by a citrullyl residue. A “fibrin-derived peptide having at least one citrullyl residue” according to the invention means a peptide recognised by the ACPAs, and can also be denoted a “citrullinated peptide”. Such peptides can be obtained from natural, recombinant or synthetic fibrin or fibrinogen fragments. Such peptides can also be obtained by direct synthesis. The amino acids constituting the peptide can be L or D amino acids, preferably L. Said substitution can be carried out for example by an enzymatic deimination step under the action of peptidyl-arginine deiminases (PAD). Such a peptide can also be obtained by directly incorporating one or more citrullyl residues into the synthesised peptide. A peptide according to the invention binds to ACPAs, and the binding between said fibrin-derived peptide having at least one citrullyl residue and an ACPA can be verified using an ELISA test or as described in the publication by Sebbag M, Moinard N, Auger I, Clavel C, Arnaud J, Nogueira L, Roudier J, Serre G, Epitopes of human fibrin recognized by the rheumatoid arthritis-specific autoantibodies to citrullinated proteins. Eur J Immunol 36:2250-2263, 2006.

According to one embodiment, in said hybrid molecule according to the invention, said peptide is derived from all or part of the sequence of the α or β chain of a vertebrate fibrin, by substitution of at least one arginyl residue with a citrullyl residue. Preferably, said peptide is derived from a sequence of at least 5 consecutive amino acids of the α chain (in particular represented by SEQ ID NO: 27) or β (in particular represented by SEQ ID NO: 28), of a vertebrate fibrin. Even more particularly, said vertebrate fibrin is a mammalian, preferably human, fibrin.

According to one embodiment, in said hybrid molecule according to the invention, the peptide has a size of at least 2 consecutive amino acids, 3 consecutive amino acids, 4 consecutive amino acids, preferably 5 consecutive amino acids, and even more preferably between 5 and 25 amino acids. According to the invention, “between 5 and 25” means all the values: 5, 6, 7, 8, 9, 10, 11, 12, 13, 14, 15, 16, 17, 18, 19, 20, 21, 22, 23, 24 and 25. Preferably, said peptide has a size of between 10 and 20 amino acids, more particularly 15 amino acids.

According to one embodiment, in said hybrid molecule according to the invention, said peptide is preferably linear.

According to one embodiment, in said hybrid molecule according to the invention, the peptide can be modified so as to improve its reactivity to ACPAs. By way of example, the peptides can be cyclised, the peptides can be retro peptides (L amino acids are concatenated according to a reverse sequence of the peptide to be reproduced), or retro-inverso peptides (the amino acids D amino acids instead of the natural L amino acids and are concatenated according to a reverse sequence of the peptide to be reproduced). According to an even more particular embodiment, in said hybrid molecule according to the invention, the terminal carboxyl function (COOH) of said peptide is replaced by a carboxamide function (CONH₂). Preferably, in the peptide of SEQ ID NO: 12, the terminal carboxyl function (COOH) of said peptide is replaced by a carboxamide function (CONH₂). By way of example, the β60-74Cit peptide (as represented in SEQ ID NO:19) advantageously has such a carboxamide function.

According to another embodiment, in said hybrid molecule according to the invention, the peptide can be modified so as to facilitate its synthesis and/or improve its stability, for example by alkylation. According to an even more particular embodiment, in said hybrid molecule according to the invention, the terminal amine function (NH₂) of said peptide is acetylated. Preferably, in the peptide of SEQ ID NO: 1, the terminal amine function of said peptide is acetylated. By way of example, the α621-635Cit peptide (as shown in SEQ ID NO: 18) advantageously has such an acetyl function on its terminal amine (NH₂).

According to one embodiment, in said hybrid molecule according to the invention, the amine and carboxyl functions of the peptide can be in the form of the salt corresponding to the acid or the base.

According to one embodiment, in said hybrid molecule according to the invention, said peptide comprises at least one citrullyl residue, and is selected from the group consisting of:
a) a peptide defined by the sequence X₁PAPPPISGGGYX₂AX₃ (SEQ ID NO: 1) in which X₁, X₂, and X₃ each represent a citrullyl residue or an arginyl residue, and at least one of the X₁ or X₂ or X₃ residues is a citrullyl residue;
b) a peptide defined by the sequence GPX₁VVEX₂HQSACKDS (SEQ ID NO: 2) in which X₁ and X₂ each represent a citrullyl residue or an arginyl residue, and at least one of the X₁ or X₂ residues is a citrullyl residue;
c) a peptide defined by the sequence SGIGTLDGFX₁HX₂HPD (SEQ ID NO: 3) in which X₁ and X₂ each represent a citrullyl residue or an arginyl residue, and at least one of the X₁ or X₂ residues is a citrullyl residue;
d) a peptide defined by the sequence VDIDIKIX₁SCX₂GSCS (SEQ ID NO: 4) in which X₁ and X₂ each represent a citrullyl residue or an arginyl residue, and at least one of the X₁ or X₂ residues is a citrullyl residue;
e) a peptide defined by the sequence X₁GHAKSX₂PVX₃GIHTS (SEQ ID NO: 12) in which X₁, X₂ and X₃ each represent a citrullyl residue or an arginyl residue, and at least one of the X₁ or X₂ or X₃ residues is a citrullyl residue;
f) a peptide comprising at least 5 consecutive amino acids, including at least one citrullyl residue, from one of peptides a) to e) above.

According to one particular embodiment, said peptide comprises at least one citrullyl residue, and is selected from the group consisting of:
a peptide defined by sequence SEQ ID NO: 1 in which at least one residue selected from X₁ or X₂ or X₃ is a citrullyl residue, or a peptide comprising a fragment of at least 5 consecutive amino acids of said sequence containing said citrullyl residue(s);
a peptide defined by sequence SEQ ID NO: 2 in which at least X₁ or X₂ is a citrullyl residue, or a peptide comprising a fragment of at least 5 consecutive amino acids of said sequence containing said citrullyl residue(s);
a peptide defined by sequence SEQ ID NO: 3 in which at least X₁ or X₂ is a citrullyl residue, or a peptide comprising a fragment of at least 5 consecutive amino acids of said sequence containing said citrullyl residue or residues;
a peptide defined by sequence SEQ ID NO: 4 in which at least X₁ or X₂ is a citrullyl residue, or a peptide comprising a fragment of at least 5 consecutive amino acids of said sequence containing said citrullyl residue or residues;
a peptide defined by sequence SEQ ID NO: 12 in which at least X₁ or X₂ or X₃ is a citrullyl residue, or a peptide comprising a fragment of at least 5 consecutive amino acids of said sequence containing said citrullyl residue or residues.

According to an even more particular embodiment, in said hybrid molecule according to the invention, said peptide comprises at least one citrullyl residue, and is selected from the group consisting of:
a peptide defined by sequence SEQ ID NO: 1 in which X₁, X₂, and X₃ are citrullyl residues, or a peptide of at least 15 amino acids comprising said sequence (which is a peptide of SEQ ID NO: 19);
a peptide defined by sequence SEQ ID NO: 2 in which X₁ and X₂ are citrullyl residues, or a peptide comprising a fragment of at least 5 consecutive amino acids of said sequence containing said citrullyl residues (which is a peptide of SEQ ID NO: 5);
a peptide defined by sequence SEQ ID NO: 3 in which X₁ and X₂ are citrullyl residues, or a peptide comprising a fragment of at least 5 consecutive amino acids of said sequence containing said citrullyl residues (which is a peptide of SEQ ID NO: 14);
a peptide defined by sequence SEQ ID NO: 4 in which X₁ and X₂ are citrullyl residues, or a peptide comprising a fragment of at least 5 consecutive amino acids of said sequence containing said citrullyl residues (which is a peptide of SEQ ID NO: 6);
a peptide defined by sequence SEQ ID NO: 12 in which X₁, X₂ and X₃ are citrullyl residues, or a peptide comprising a fragment of at least 10 consecutive amino acids of said sequence containing said citrullyl residues (which is a peptide of SEQ ID NO: 18).

According to an even more particular embodiment, in said hybrid molecule according to the invention, said peptide is selected from the group consisting of: SEQ ID NO: 5 (α36-50cit_{38,42}), SEQ ID NO: 6 (α171-185cit_{178,181}), SEQ ID NO: 7 (α183-197cit_{186,190}), SEQ ID NO: 8 (α246-260cit₂₅₈), SEQ ID NO: 9 (α259-273cit_{263,271}), SEQ ID NO: 10 (α366-380cit₃₆₇), SEQ ID NO: 11 (α396-410cit₄₀₄), SEQ ID NO: 13 (α411-425cit₄₂₅), SEQ ID NO: 14 (α501-515cit_{510,512}), SEQ ID NO: 15 (α546-560cit₅₄₇), SEQ ID NO: 16 (α561-575cit₅₇₃), SEQ ID NO: 17 (α588-602cit₅₉₁), SEQ ID NO: 18 (α621-635cit_{621,627,630}), SEQ ID NO: 19 (β60-74cit_{60,72,74}), SEQ ID NO: 20 (β210-224cit₂₂₄), SEQ ID NO: 21 (β281-295cit_{285,294}), SEQ ID NO: 22 (3420-434cit₄₂₁) and SEQ ID NO: 23 (β433-447cit_{436,445}), more particularly selected from the group consisting of: SEQ ID NO: 5 (α36-50cit_{38,42}), SEQ ID NO: 6 (α171-185cit_{178,181}), SEQ ID NO: 14 (α501-515cit_{510,512}), SEQ ID NO: 18 (α621-635cit_{621,627,630}) and SEQ ID NO: 19 (β60-74cit_{60,72,74}), even more particularly selected from SEQ ID NO: 18 (α621-635cit_{621,627,630}) and SEQ ID NO: 19 (β60-74cit_{60,72,74}).

The antibody or antibody fragment and the peptide each have N- and C-termini. The antibody or antibody fragment can therefore be bound via its N- or C-terminus to the N- or C-terminus of the peptide. According to another preferred embodiment, in said hybrid molecule according to the invention, said covalent bond is located between the N-terminus of said peptide and the N-terminus of said antibody or antibody fragment, or between the N-terminus of said peptide and the C-terminus of said antibody or antibody fragment. According to one embodiment, when a spacer is present, the spacer can be bound to the antibody or antibody fragment via its N- or C-terminus. According to another embodiment, when two spacers are present, the first spacer can be bound to the antibody or antibody fragment via its N- or C-terminus and the second spacer can be bound to the peptide via its N- or C-terminus, in particular the N-terminus. Alternatively, said covalent bond between said antibody or antibody fragment and said peptide (optionally in the presence of one or more spacers) can be created on all or part of the antibody or antibody fragment, provided that the latter retains its capacity to bind plasma cells. According to the invention, “all or part of the antibody or fragment” means that different amino acids that constitute the antibody or the fragment can be involved in a covalent bond with said peptide.

According to a preferred embodiment, when at least one spacer is present in said hybrid molecule according to the invention, said spacer enables binding of the antibody or fragment to an azide or an alkyne that will itself be involved in the covalent bond with said peptide. According to one embodiment, when at least one spacer is present in said hybrid molecule according to the invention, said spacer enables binding of the peptide to an alkyne or an azide that will itself be involved in the covalent bond with the antibody or fragment. According to an even more preferred embodiment, the hybrid molecule according to the invention comprises at least two spacers: a first spacer which enables binding of the antibody or fragment to an azide or an alkyne and a second spacer which enables binding of the peptide to an azide (when the antibody or fragment is bound to an alkyne) or an alkyne (when the Fc fragment is bound to an azide).

According to one embodiment according to the invention, in said hybrid molecule according to the invention, said antibody or said fragment:
is coupled to an azide or an alkyne, such as a cyclooctyne, and in particular DBCO, or
is bound to a spacer that is itself coupled to an azide or an alkyne, such as a cyclooctyne, and in particular DBCO, and said peptide is:
either coupled with an azide or an alkyne, such as a cyclooctyne, and in particular DBCO,
or bound to a spacer that is itself coupled to an azide or an alkyne, such as a cyclooctyne, and in particular DBCO,
   the covalent bond between said fragment and said peptide, optionally in the presence of one or more spacers, being created between the azide and the alkyne.

According to one embodiment according to the invention, in said hybrid molecule according to the invention:
said antibody or said fragment is coupled to at least one azide and said peptide is coupled to an alkyne, such as a cyclooctyne, and in particular DBCO, or
said antibody or said fragment is coupled to at least one alkyne, such as a cyclooctyne, and in particular DBCO, and said peptide is coupled to an azide,
   the covalent bond between said antibody or said fragment and said peptide being created between the azide and the alkyne.

According to one embodiment according to the invention, in said hybrid molecule according to the invention:
said antibody or said fragment is bound to a spacer, itself coupled to an azide, and said peptide is coupled to an alkyne such as a cyclooctyne, and in particular DBCO, or
said antibody or said fragment is bound to a spacer, itself coupled to an alkyne such as a cyclooctyne, and in particular DBCO, and said peptide is coupled to an azide,
   the covalent bond between said antibody or said fragment and said peptide being created between the azide and the alkyne in the presence of a spacer.

According to one embodiment according to the invention, in said hybrid molecule according to the invention:
said antibody or said fragment is bound to a spacer, itself coupled to an azide, and said peptide is also bound to a second spacer, itself coupled to an alkyne such as a cyclooctyne, and in particular DBCO, or
said antibody or said fragment is bound to a spacer, itself coupled to an alkyne such as a cyclooctyne, and in particular DBCO, and said peptide is also bound to a second spacer, itself coupled to an azide,
   the covalent bond between said antibody or said fragment and said peptide being created between the azide and the alkyne in the presence of one or more spacers.

According to one embodiment, in the molecules described above, more particularly from page 9, line 15 to page 10, line 12, the spacer used is preferably a PEGn spacer, n preferably representing an integer between 1 and 10. When the hybrid molecule according to the invention comprises at least two PEGn spacers, the values of n of the two spacers can be identical or different. For example, the first spacer can be a PEG₂ and the second spacer can be a PEG₃ or PEG₄.

According to one particularly preferred embodiment, in the molecules described above, more particularly from page 9, line 15 to page 10, line 12, the peptide is derived from human fibrin or fibrinogen and the antibody or antibody fragment is human.

According to one embodiment according to the invention, said hybrid molecule according to the invention is represented by:
an antibody fragment capable of binding to CD38 bound to a PEGn spacer, preferably PEG₃, itself coupled to an azide covalently bound to a cyclooctyne coupled to a peptide represented by SEQ ID NO: 19 (β60-74cit_{60,72,74}) or SEQ ID NO: 18 (α621-635cit_{621,627,630}), or
an antibody fragment capable of binding to CD38 bound to a PEGn spacer, preferably PEG₃, itself coupled to an azide covalently bound to a cyclooctyne coupled to a PEGn spacer, preferably PEG₃, itself bound to a peptide represented by SEQ ID NO: 19 (β60-74cit_{60,72,74}), or SEQ ID NO: 18 (α621-635cit_{621,627,630}), n preferably representing an integer between 1 and 10, more particularly 1, 2, 3, 4 or 8, and said fragment preferably being an Fab fragment.

According to an even more particular embodiment according to the invention, said hybrid molecule according to the invention is represented by:
an antibody fragment capable of binding to CD38 bound to a PEGn spacer, preferably PEG₃, itself coupled to an azide covalently bound to a cyclooctyne coupled to a peptide represented by SEQ ID NO: 19 (ß60-74cit_{60,72,74}), or
an antibody fragment capable of binding to CD38 bound to a PEGn spacer, preferably PEG₃, itself coupled to an azide covalently bound to a cyclooctyne coupled to a PEGn spacer, preferably PEG₃, itself coupled to a peptide represented by SEQ ID NO: 19 (β60-74cit_{60,72,74}), or
an antibody fragment capable of binding to CD38 bound to a PEGn spacer, preferably PEG₃, itself coupled to an azide covalently bound to a cyclooctyne coupled to a PEGn spacer, preferably PEG₃, itself coupled to a peptide represented by SEQ ID NO: 18 (α621-635cit_{621,627,630}),
   n preferably representing an integer between 1 and 10, more particularly 1, 2, 3, 4 or 8, and said fragment preferably being a Fab fragment.

According to the invention, the formation of the covalent bond between the azide and the alkyne corresponds to what is known as a “click-chemistry” step, the N₃ part of the azide reacting with an alkyne. Azide means salts of hydrazoic acid HN₃, or organic azides in which one of the nitrogen atoms is covalently bound with a carbon atom of an organic compound (for example methyl azide CH₃N₃). Preferably, the azide is represented by the formula N₃. Alkyne means molecules having the general formula CnH2n-2, which are characterised by the presence of at least one triple bond. The alkyne is preferably a cyclooctyne, even more preferably dibenzocyclooctyne (DBCO).

The antibody or fragment, said peptide and optionally said spacer(s), are coupled to the alkyne or to the azide by any conventionally used molecular coupling technique (such as conjugation). Any technique may also be used to covalently bond the antibody or fragment to the spacer and/or the peptide to the spacer.

More particularly, a conjugation technique means enzymatic conjugation or chemical conjugation. Enzymatic conjugation means, for example, conjugation using a transglutaminase that catalyses the formation of covalent bonds between free amine groups and glutamine or lysine residues or even using a transpeptidase such as sortase. For more information on enzymatic conjugation, see, for example, patent applications US20160361434 or US20170313787, or the publication by Ohtsuka et al., Bioscience, Biotechnology, and Biochemistry Volume 64, 2000-Issue 12, Comparison of Substrate Specificities of Transglutaminases Using Synthetic Peptides as Acyl donors. The transglutaminase substrate is, for example, a peptide comprising a glutamyl residue (a Q-tag), as represented by SEQ ID NO: 29 (LLQG). The antibody or fragment may carry the Q-tag. Chemical conjugation means, for example, a covalent bond between a cysteine which is isolated or participating in a disulfide bridge after reduction of the latter and, for example, a maleimide.

According to the invention, the term “coupled” or “molecular coupling” means the establishment of a covalent bond, thus the antibody or fragment and/or the peptide and/or the spacer are covalently bound to a alkyne or an azide. The term “bound” also means a covalent bond. Thus, by way of example, the expression “said antibody or said fragment is bound to a spacer, itself coupled to an azide, and said peptide is also bound to a second spacer, itself coupled to an alkyne such as a cyclooctyne”, can also read “said antibody or fragment is covalently bound to a spacer, itself covalently bound to an azide, and said peptide is also covalently bound to a second spacer, itself covalently bound to an alkyne such as cyclooctyne”.

### Use of the Hybrid Molecules According to the Invention

In a second aspect, the present invention also relates to a hybrid molecule as defined above for use as a medicinal product.

More particularly, according to the invention, said hybrid molecules are intended to target and lyse the ACPA-secreting plasma cells inside the patients' bodies, by apoptosis, by phagocytosis, by ADCC or by complement activation. This is due to the hybrid molecule according to the invention binding to the plasma cells thanks to the antibody or fragment which binds to CD38 and/or CD138. The hybrid molecule according to the invention also binds to ACPAs thanks to the fibrin-derived peptide that has a citrullyl residue, this being the target of said ACPAs. ACPAs will allow bridging of the hybrid molecules according to the invention bound to the surface of the plasma cells and thus induce their apoptosis. ACPAs, like any antibody, have effector functions that are activated after binding to their target antigen, thus participating in the destruction of plasma cells by phagocytosis and/or by ADCC and/or by complement activation.

According to one particular embodiment, the invention relates to a hybrid molecule, as defined above, for the use thereof in the treatment of autoimmune diseases associated with the production of autoantibodies against citrullinated proteins, in particular Gougerot-Sjögren syndrome and rheumatoid arthritis. In this embodiment, the severe forms of said autoimmune diseases associated with the production of anti-citrullinated protein autoantibodies are targeted, as well as the forms “bordering” on other chronic arthritis, such as psoriatic arthritis or systemic lupus erythematosus.

According to one embodiment, the invention also relates to a pharmaceutical composition comprising a hybrid molecule according to any one of the preceding claims, in combination with a pharmaceutically acceptable vehicle.

According to the invention, “a pharmaceutically acceptable vehicle” means any formulation that makes the composition suitable for administration to a patient, in any galenic form.

As indicated above, the present invention aims to target ACPA-secreting plasma cells. However, ACPA-positive B lymphocytes, precursors of plasma cells, before their differentiation, are not, or only slightly, targeted by said hybrid molecules according to the invention. According to one particular embodiment, the present invention thus relates to a hybrid molecule as previously described for the use thereof as a medicinal product in combination with a second hybrid molecule, said second hybrid molecule comprising at least one antibody Fc fragment covalently bound to at least one fibrin-derived peptide carrying at least one citrullyl residue, a spacer being optionally present between said Fc fragment and said peptide. Such a combination therapy makes it possible to specifically target and destroy ACPA-secreting clonal plasma cells and ACPA-positive B lymphocytes, which are the precursors thereof, and thus cause the disappearance of the ACPAs from the patients' bodies and prevent their reappearance.

According to one particular embodiment, in said second hybrid molecule according to the invention, said Fc fragment is a human Fc fragment, in particular of IgG, more particularly of IgG1. IgG1 can correspond to any of its allotypes, for example G1m3 or nG1m1. By way of example, the Fc fragment of IgG1 is represented by SEQ ID NO: 24, SEQ ID NO: 25 (Fc+Q-tag) or SEQ ID NO: 26 (Fc+Q-tag bis).

According to one embodiment, in said second hybrid molecule according to the invention, said Fc fragment is wild-type or mutated. The mutation(s) may aim to increase or reduce the plasma half-life or modify the effector functions of the Fc fragment etc. . . .

According to an even more particular embodiment, said mutated Fc fragment comprises at least the following mutations:
L234A et L235A (LALA), or
L234A, L235A et P329G (LALAPG), or
G236A, S239D and 1332E (GASDIE), or
G236A, S239D, A330L and 1332E (GASDALIE), or
S239D, H268F, S324T and 1332E (SDHFSTIE or SDH),
   the numbering being indicated in the sequence of a human IgG1 according to the EU index. Such mutations are described in the article by Bruhns and Jonsson, Immunol Rev. 2015 November; 268(1):25-51. Preferably, when the hybrid molecule is used in therapy, said mutated Fc fragment comprises at least the GASDIE, GASDALIE, or SDH mutations.

According to one particular embodiment, in said second hybrid molecule according to the invention, said Fc fragment has a fucosylation rate of between 0% and 100% of the glycosylated forms. According to the invention “between 0% and 100%” represents all the integer values between 0 and 100, i.e. 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%. Weak fucosylation of the Fc fragment causes a strong ADCC response. This is why according to one particular embodiment said Fc fragment has a fucosylation rate of the glycosylated forms of between 0% and 60%, in particular 50%, 40%, 30%, 20%, 10% or 0%. According to the invention, the fucosylation rate is defined as the average proportion of fucose carried by the Fc fragment relative to the maximum quantity of fucose that an Fc fragment can carry.

According to this particular embodiment of the invention, the first and second hybrid molecules are administered simultaneously, separately or gradually over time.

According to another embodiment, the hybrid molecule according to the invention can be coupled to at least one radioisotope or to at least one fluorochrome, such as A488 or A647. Such molecules can advantageously be used as molecular tracer tools.

According to another embodiment, the invention thus relates to in vitro or ex vivo use as a molecular tool of a hybrid molecule comprising at least one fibrin-derived peptide having at least one citrullyl residue, said peptide being covalently bound to at least one antibody or to at least one antibody fragment, said antibody or fragment being capable of binding to CD38 and/or CD138, one or more spacers being optionally present between said peptide and said antibody or said fragment. Such constructs can in particular be used to analyse the binding of hybrid molecules to ACPAs and to plasma cells, as well as to analyse the reactivity of plasma cells to the binding of hybrids followed by their bridging to the ACPAs, etc. . . . The radioisotopes and/or fluorochromes are preferably coupled to the antibody fragment.

### Method for Producing the Hybrid Molecules According to the Invention

In another aspect, the invention also relates to a method that enables obtaining an immune cell grafted with a hybrid molecule as defined previously.

According to one embodiment, the invention thus relates to a method for producing a hybrid molecule as defined above, comprising the following steps:
(i) obtaining an azide coupled to an antibody or to a fragment capable of binding to CD38 and/or CD138, or obtaining an alkyne coupled to an antibody or to a fragment capable of binding to CD38 and/or CD138,
(ii) obtaining an alkyne coupled to a peptide or obtaining an azide coupled to a peptide,
(iii) mixing the products generated in (i) and (ii), the covalent bond being established specifically between the azide and the alkyne,
it being possible to carry out step (i) before or after step (ii), or else at the same time.

According to one embodiment, the invention thus relates to a method for producing a hybrid molecule as defined above, comprising the following steps:
(i) coupling an azide to an antibody or to a fragment capable of binding to CD38 and/or CD138, optionally in the presence of a spacer,
(ii) coupling an alkyne to a peptide, optionally in the presence of a spacer,
(iii) mixing the products generated in (i) and (ii), the covalent bond being established specifically between the azide and the alkyne.
it being possible to carry out step (i) before or after step (ii), or else at the same time.

According to another embodiment, the invention also relates to a method for producing a hybrid molecule as defined above, comprising the following steps:
(i) coupling an alkyne to an antibody or to a fragment capable of binding to CD38 and/or CD138, optionally in the presence of a spacer,
(ii) coupling an azide to a peptide, optionally in the presence of a spacer,
(iii) mixing the products generated in (i) and (ii), the covalent bond being established specifically between the azide and the alkyne, it being possible to carry out step (i) before or after step (ii), or else at the same time.

According to another embodiment, the invention also relates to a method for producing a hybrid molecule as defined above, comprising the following steps:
(i) coupling at least one alkyne or azide to each monomer of the antibody or the fragment capable of binding to CD38 and/or CD138, optionally in the presence of a spacer,
(ii) coupling at least one azide or alkyne to a peptide, optionally in the presence of a spacer,
(iii) mixing the products generated in (i) and (ii), the covalent bond being established specifically between the azide and the alkyne, it being possible to carry out step (i) before or after step (ii), or else at the same time.

The sequences of the invention are shown in Table 1 below.

TABLE 1
Summary table of the sequences of the invention

| Sequence | |
|---|---|
| number/sequence | |
| name if applicable | Sequence |
| SEQ ID NO: 1 | X₁PAPPPISGGGYX₂AX₃ |
| SEQ ID NO: 2 | GPX₁VVEX₂HQSACKDS |
| SEQ ID NO: 3 | SGIGTLDGFX₁HX₂HPD |
| SEQ ID NO: 4 | VDIDIKIX₁SCX₂GSCS |
| SEQ ID NO: 12 | X₁GHAKSX₂PVX₃GIHTS |
| SEQ ID NO: 5/ | GPXVVEXHQSACKDS, X being a citrullyl residue |
| α36-50cit_{38,42} | |
| SEQ ID NO: 6/ | VDIDIKIXSCXGSCS, X being a citrullyl residue |
| α171-185cit_{178,181} | |
| SEQ ID NO: 7/ | SCSXALAXEVDLKDY, X being a citrullyl residue |
| α183-197cit_{186,190} | |
| SEQ ID NO: 7/ | PEWKALTDMPQMXME, X being a citrullyl residue |
| α246-260cit₂₅₈ | |
| SEQ ID NO: 9/ | MELEXPGGNEITXGG, X being a citrullyl residue |
| α259-273cit_{263,271} | |
| SEQ ID NO: 10/ | EXGSAGHWTSESSVS, X being a citrullyl residue |
| α366-380cit₃₆₇ | |
| SEQ ID NO: 11/ | DSPGSGNAXPNNPDW, X being a citrullyl residue |
| α396-410cit₄₀₄ | |
| SEQ ID NO: 13/ | GTFEEVSGNVSPGTX, X being a citrullyl residue |
| α411-425cit₄₂₅ | |
| SEQ ID NO: 14/ | SGIGTLDGFXHXHPD, X being a citrullyl residue |
| α501-515cit_{510,512} | |
| SEQ ID NO: 15/ | SXGSESGIFTNTKES, X being a citrullyl residue |
| α546-560cit₅₄₇ | |
| SEQ ID NO: 16/ | SSHHPGIAEFPSXGK, X being a citrullyl residue |
| α561-575cit₅₇₃ | |
| SEQ ID NO: | SYNXGDSTFESKSYK, X being a citrullyl residue |
| 17/α588-602cit₅₉₁ | |
| SEQ ID NO: 18/ | XGHAKSXPVXGIHTS, X being a citrullyl residue |
| α621- | |
| 635cit_{621,627,630} | |
| SEQ ID NO: 19/ | XPAPPPISGGGYXAX, X being a citrullyl residue |
| ß60-74cit_{60,72,74} | |
| SEQ ID NO: 20/ | QKLESDVSAQMEYCX, X being a citrullyl residue |
| β210-224cit₂₂₄ | |
| SEQ ID NO: 21/ | VIQNXQDGSVDFGXK, X being a citrullyl residue |
| (281-295cit_{285,294} | |
| SEQ ID NO: 22/ | PXKQCSKEDGGGWWY, X being a citrullyl residue |
| β420-434cit₄₂₁ | |
| SEQ ID NO: 23/ | WYNXCHAANPNGXYY, X being a citrullyl residue |
| β433-447cit_{436,445} | |
| SEQ ID NO: 27/ | GPRVVERHQSACKDSDWPFCSDEDWNYKCPSGCRMKGLID |
| Fibrin α chain | EVNQDFTNRINKLKNSLFEYQKNNKDSHSLTTNIMEILRG |
| | DFSSANNRDNTYNRVSEDLRSRIEVLKRKVIEKVQHIQLL |
| | QKNVRAQLVDMKRLEVDIDIKIRSCRGSCSRALAREVDLK |
| | DYEDQQKQLEQVIAKDLLPSRDRQHLPLIKMKPVPDLVPG |
| | NFKSQLQKVPPEWKALTDMPQMRMELERPGGNEITRGGST |
| | SYGTGSETESPRNPSSAGSWNSGSSGPGSTGNRNPGSSGT |
| | GGTATWKPGSSGPGSTGSWNSGSSGTGSTGNQNPGSPRPG |
| | STGTWNPGSSERGSAGHWTSESSVSGSTGQWHSESGSFRP |
| | DSPGSGNARPNNPDWGTFEEVSGNVSPGTRREYHTEKLVT |
| | SKGDKELRTGKEKVTSGSTTTTRRSCSKTVTKTVIGPDGH |
| | KEVTKEVVTSEDGSDCPEAMDLGTLSGIGTLDGFRHRHPD |
| | EAAFFDTASTGKTFPGFFSPMLGEFVSETESRGSESGIFT |
| | NTKESSSHHPGIAEFPSRGKSSSYSKQFTSSTSYNRGDST |
| | FESKSYKMADEAGSEADHEGTHSTKRGHAKSRPVRGIHTS |
| SEQ ID NO: 28/ | GHRPLDKKREEAPSLRPAPPPISGGGYRARPAKAAATQKK |
| Fibrin β chain | VERKAPDAGGCLHADPDLGVLCPTGCQLQEALLQQERPIR |
| | NSVDELNNNVEAVSQTSSSSFQYMYLLKDLWQKRQKQVKD |
| | NENVVNEYSSELEKHQLYIDETVNSNIPTNLRVLRSILEN |
| | LRSKIQKLESDVSAQMEYCRTPCTVSCNIPVVSGKECEEI |
| | IRKGGETSEMYLIQPDSSVKPYRVYCDMNTENGGWTVIQN |
| | RQDGSVDFGRKWDPYKQGFGNVATNTDGKNYCGLPGEYWL |
| | GNDKISQLTRMGPTELLIEMEDWKGDKVKAHYGGFTVQNE |
| | ANKYQISVNKYRGTAGNALMDGASQLMGENRTMTIHNGMF |
| | FSTYDRDNDGWLTSDPRKQCSKEDGGGWWYNRCHAANPNG |
| | RYYWGGQYTWDMAKHGTDDGVVWMNWKGSWYSMRKMSMKI |
| | RPFFPQQ |
| SEQ ID NO: 24/ | APELLGGPSVFLFPPKPKDTLMISRTPEVTCVVVDVSHED |
| Fc Fragment | PEVKFNWYVDGVEVHNAKTKPREEQYNSTYRVVSVLTVLH |
| | QDWLNGKEYKCKVSNKALPAPIEKTISKAKGQPREPQVYT |
| | LPPSREEMTKNQVSLTCLVKGFYPSDIAVEWESNGQPENN |
| | YKTTPPVLDSDGSFFLYSKLTVDKSRWQQGNVFSCSVMHE |
| | ALHNHYTQKSLSLSPGK |
| SEQ ID NO: 25/ | LLQGARSDATHTCPPCPAPELLGGPSVFLFPPKPKDTLMI |
| Fc Fragment + Q- | SRTPEVTCVVVDVSHEDPEVKFNWYVDGVEVHNAKTKPRE |
| tag | EQYNSTYRVVSVLTVLHQDWLNGKEYKCKVSNKALPAPIE |
| | KTISKAKGQPREPQVYTLPPSREEMTKNQVSLTCLVKGFY |
| | PSDIAVEWESNGQPENNYKTTPPVLDSDGSFFLYSKLTVD |
| | KSRWQQGNVFSCSVMHEALHNHYTQKSLSLSPGK |
| SEQ ID NO: 26/ | AHGHGHGLLQGARSDATHTCPPCPAPELLGGPSVFLFPPK |
| Fc Fragment + Q- | PKDTLMISRTPEVTCVVVDVSHEDPEVKFNWYVDGVEVHN |
| tag bis | AKTKPREEQYNSTYRVVSVLTVLHQDWLNGKEYKCKVSNK |
| | ALPAPIEKTISKAKGQPREPQVYTLPPSREEMTKNQVSLT |
| | CLVKGFYPSDIAVEWESNGQPENNYKTTPPVLDSDGSFFL |
| | YSKLTVDKSRWQQGNVFSCSVMHEALHNHYTQKSLSLSPG |
| | K |
| SEQ ID NO: 29 | LLQG |
| (Example of Q- | |
| tag) | |
| SEQ ID NO: 30 | MFSMRIVCLVLSVVGTAWTADSGEGDFLAEGGGVRGPRVV |
| Fibrinogen α | ERHQSACKDSDWPFCSDEDWNYKCPSGCRMKGLIDEVNQD |
| chain | FTNRINKLKNSLFEYQKNNKDSHSLTTNIMEILRGDFSSA |
| | NNRDNTYNRVSEDLRSRIEVLKRKVIEKVQHIQLLQKNVR |
| | AQLVDMKRLEVDIDIKIRSCRGSCSRALAREVDLKDYEDQ |
| | QKQLEQVIAKDLLPSRDRQHLPLIKMKPVPDLVPGNFKSQ |
| | LQKVPPEWKALTDMPQMRMELERPGGNEITRGGSTSYGTG |
| | SETESPRNPSSAGSWNSGSSGPGSTGNRNPGSSGTGGTAT |
| | WKPGSSGPGSTGSWNSGSSGTGSTGNQNPGSPRPGSTGTW |
| | NPGSSERGSAGHWTSESSVSGSTGQWHSESGSFRPDSPGS |
| | GNARPNNPDWGTFEEVSGNVSPGTRREYHTEKLVTSKGDK |
| | ELRTGKEKVTSGSTTTTRRSCSKTVTKTVIGPDGHKEVTK |
| | EVVTSEDGSDCPEAMDLGTLSGIGTLDGFRHRHPDEAAFF |
| | DTASTGKTFPGFFSPMLGEFVSETESRGSESGIFTNTKES |
| | SSHHPGIAEFPSRGKSSSYSKQFTSSTSYNRGDSTFESKS |
| | YKMADEAGSEADHEGTHSTKRGHAKSRPVRGIHTS |
| SEQ ID NO: 31 | MKRMVSWSFHKLKTMKHLLLLLLCVFLVKSQGVNDNEEGF |
| Fibrinogen β | FSARGHRPLDKKREEAPSLRPAPPPISGGGYRARPAKAAA |
| chain | TQKKVERKAPDAGGCLHADPDLGVLCPTGCQLQEALLQQE |
| | RPIRNSVDELNNNVEAVSQTSSSSFQYMYLLKDLWQKRQK |
| | QVKDNENVVNEYSSELEKHQLYIDETVNSNIPTNLRVLRS |
| | ILENLRSKIQKLESDVSAQMEYCRTPCTVSCNIPVVSGKE |
| | CEEIIRKGGETSEMYLIQPDSSVKPYRVYCDMNTENGGWT |
| | VIQNRQDGSVDFGRKWDPYKQGFGNVATNTDGKNYCGLPG |
| | EYWLGNDKISQLTRMGPTELLIEMEDWKGDKVKAHYGGFT |
| | VQNEANKYQISVNKYRGTAGNALMDGASQLMGENRTMTIH |
| | NGMFFSTYDRDNDGWLTSDPRKQCSKEDGGGWWYNRCHAA |
| | NPNGRYYWGGQYTWDMAKHGTDDGVVWMNWKGSWYSMRKM |
| | SMKIRPFFPQQ |

Other characteristics, details and advantages of the invention will be apparent from reading the appended figures and the examples that illustrate the invention and in no way intend to limit it. In the hybrid molecules exemplified, it is the N-terminus of the Fab fragment that is bound to a PEGn spacer, and it is always the N-terminus of said peptide that is bound to the cyclooctyne, DBCO, or to a second PEGn spacer if present. Moreover, in the exemplified hybrid molecules, in the PEGn spacer bound to the Fab fragment, n represents more particularly 3 or 4, and in the PEGn spacer bound to the peptide, the PEGn spacer represents 3 or 8, although any other value of n can be used, in particular between 1 and 10.

### BRIEF DESCRIPTION OF THE FIGURES

FIG. 1

FIG. 1A shows a plasma cell that expresses at its surface the CD38 molecules to which are linked a Dara-Fab-citrullinated peptide hybrid molecule according to the invention (a Fab fragment of the Daratumumab antibody covalently bound to a citrullinated peptide).

Said hybrid molecule is itself bound by the citrullinated peptide to an ACPA. Bridging of Dara-Fab-citrullinated peptide hybrid molecules by ACPAs is cytotoxic and leads to the death of the plasma cell.

FIG. 1B shows phagocytosis by macrophages (ADP) or NK cell-induced cytotoxicity (ADCC) of a CD38+ plasma cell after ACPA bridging of Dara-Fab-citrullinated peptide hybrid molecules on the surface of a plasma cell.

A plasma cell expresses at its surface the CD38 molecules to which are bound a Dara-Fab-citrullinated peptide hybrid molecule according to the invention, said hybrid molecule itself being bound by the citrullinated peptide to an ACPA. Binding of the Fc fragment of the ACPAs to different FcγRs expressed on the surface of a macrophage or to the FcγRIIIA expressed on the surface of NK cells activates phagocytosis (ADP) and/or antibody-induced cytotoxicity (ADCC), leading to specific destruction of the plasma cell.

FIG. 2

FIG. 2 shows the diagram of an example of a hybrid molecule according to the invention.

A spacer (which is optional) is shown between the antibody or antibody fragment which binds to CD38 (Dara) and the fibrin-derived peptide which has a citrullyl residue (referred to as “citrullinated peptide”). A construct with an F(ab)′2 fragment is also shown. For example, F(ab)′2 can bind to both CD38 and CD138. Fab: Fragment Antigen Binding. Dara Fab: Fab of a specific antibody for the CD38 molecule (Daratumumab).

FIG. 3

FIG. 3 shows the reactivity of several hybrid molecules comprising the peptide of SEQ ID NO: 19 (β60-74cit_{60,72,74}) against purified anti-β60-74 ACPAs.

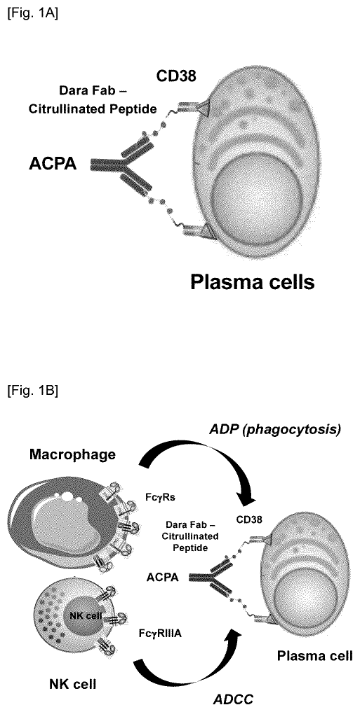

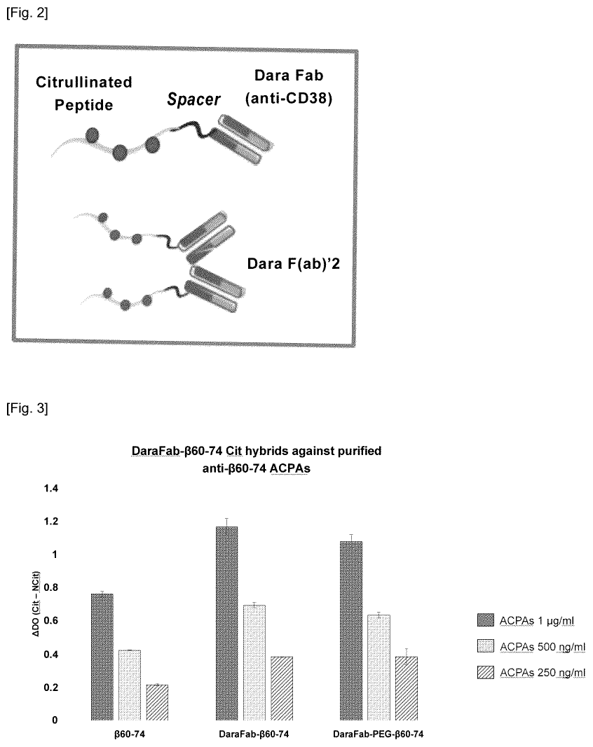

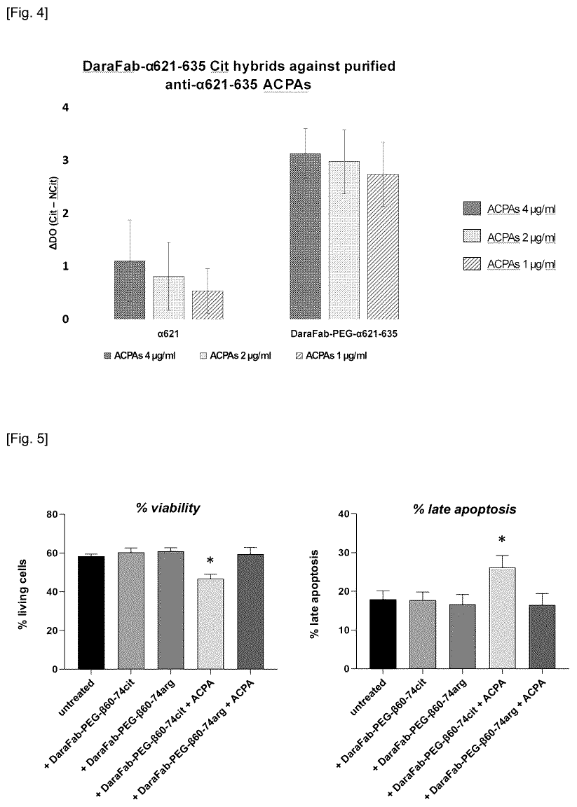

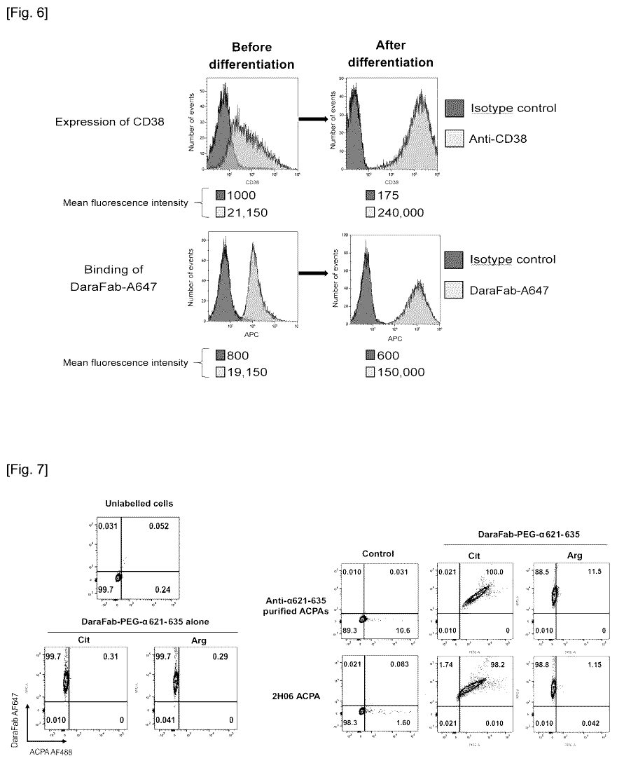

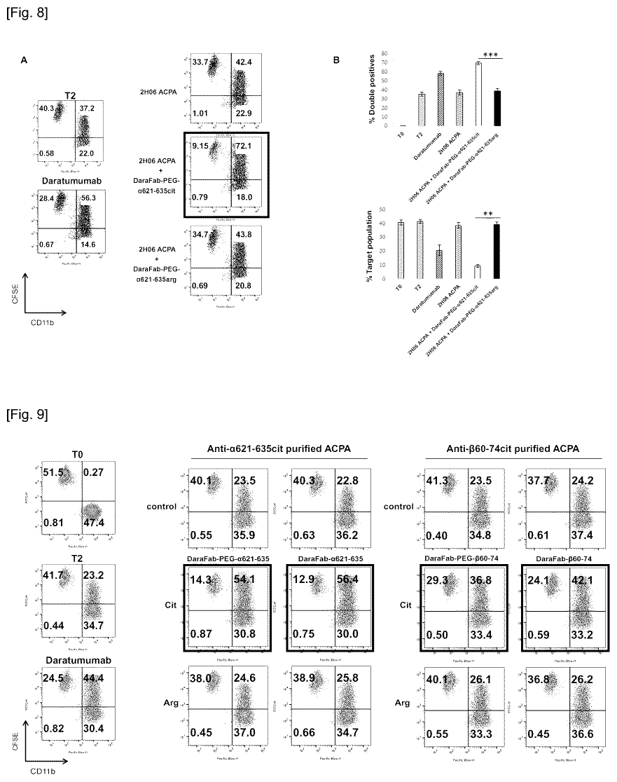

β60-74 represents the peptide of SEQ ID NO: 19. DaraFab-β60-74 represents an Fab capable of binding to CD38 bound to a PEGn spacer, itself coupled to an azide that is covalently bound to a cyclooctyne (DBCO) that is coupled to a peptide of SEQ ID NO: 19. DaraFab-PEG-β60-74 represents an Fab capable of binding to CD38 bound to a PEGn spacer, itself coupled to an azide that is covalently bound to a cyclooctyne (DBCO) that is coupled to a PEGn spacer that is itself bound to a peptide of SEQ ID NO: 19. n represents a number between 1 and 10 when a PEGn spacer is used, preferably 1, 2, 3, 4 or 8.

FIG. 4

FIG. 4 shows the reactivity of several hybrid molecules comprising the peptide of SEQ ID NO: 18 (α621) against anti-α621-635 purified ACPAs.

α621 represents the peptide of SEQ ID NO: 18. DaraFab-PEG-α621-635 represents an Fab capable of binding to CD38 bound to a PEGn spacer, itself coupled to an azide that is covalently bound to a cyclooctyne (DBCO) that is coupled to a PEGn spacer that is itself bound to a peptide of SEQ ID NO: 18. n represents a number between 1 and 10, preferably 1, 2, 3, 4 or 8.

FIG. 5

FIG. 5 shows the cytotoxicity of DaraFab-PEG-β60-74Cit against the BAEV B cell line (BC-9) produced from the lymphocytes of a patient with rheumatoid arthritis, after bridging by purified anti-β60-74 ACPAs.

The left-hand part represents the percentage of living cells, and the right-hand part the percentage of cells in late apoptosis.

FIG. 6

FIG. 6 shows the expression of CD38 and the binding of the Fab Dara (Fab fragment which binds to CD38 labelled with fluorochrome A647) on BC-9 cells before (on the left) and after (on the right) differentiation.

FIG. 7

FIG. 7 shows the interaction of DaraFab-PEG-α621-635 and purified anti-α621-635cit ACPAs or 2H06 ACPA on the surface of the KMS-12-PE myeloma cell line.

DaraFab-PEG-α621-635 represents an Fab capable of binding to CD38 bound to a PEGn spacer, itself coupled to an azide that is covalently bound to a cyclooctyne (DBCO) that is coupled to a PEGn spacer that is itself bound to a peptide of SEQ ID NO: 18. n represents a number between 1 and 10, preferably 1, 2, 3, 4 or 8.

FIG. 8

FIG. 8 shows phagocytosis of the differentiated KMS-12-PE myeloma cell line by macrophages in the presence of DaraFab-PEG-α621-635 Cit hybrids and 2H06 ACPA.

DaraFab-PEG-α621-635cit represents the hybrid molecule as described in FIG. 7. DaraFab-PEG-α621-635arg represents an Fab capable of binding to CD38 bound to a PEGn spacer, itself coupled to an azide that is covalently bound to a cyclooctyne (DBCO) that is coupled to a PEGn spacer that is itself bound to a peptide of SEQ ID NO: 18, in which X represents arginyl residues (i.e. the peptide is not citrullinated).

FIG. 9

FIG. 9 shows phagocytosis of the differentiated KMS-12-PE myeloma cell line by macrophages in the presence of DaraFab-PEG-β60-74 or DaraFab-PEG-α621-635 and, respectively, purified anti-β60-74cit or anti-α621-635cit ACPAs.

DaraFab-PEG-α621-635 is the molecule as depicted in FIG. 7. DaraFab-α621-635 represents an Fab capable of binding to CD38 bound to a PEGn spacer, itself coupled to an azide that is covalently bound to a cyclooctyne (DBCO) that is coupled to a peptide of SEQ ID NO: 18. DaraFab-PEG-β60-74 represents an Fab capable of binding to CD38 bound to a PEGn spacer, itself coupled to an azide that is covalently bound to a cyclooctyne (DBCO) that is coupled to a PEGn spacer that is itself bound to a peptide of SEQ ID NO: 19 (n represents a number between 1 and 10, preferably 1, 2, 3, 4 or 8). DaraFab-β60-74 represents an Fab capable of binding to CD38 bound to a PEGn spacer, itself coupled to an azide that is covalently bound to a cyclooctyne (DBCO) that is coupled to a peptide of SEQ ID NO: 19.

FIG. 10

FIG. 10 represents the induction of apoptosis of the differentiated KMS-12-PE myeloma cell line by NK cells in the presence of DaraFab-PEG-α621-635 and of purified anti-α621-635cit ACPA or 2H06 ACPA.

The hybrid molecules are those described in FIG. 8.

### EXAMPLES

### Example 1: Example of Production of a Hybrid Molecule According to the Invention

The hybrid molecule described here comprises the following construct: an Fab fragment covalently bound to a PEGn, itself coupled to an azide, said azide being covalently bound to an alkyne that is itself bound to a spacer coupled to a citrullinated peptide. Such a molecule can be read: Fab-PEGn-N₃-DBCO-PEGn-citrullinated peptide.

1. Synthesis of an NH₂-PEGn-N₃ (i.e. a spacer coupled to an azide at one end and having a free amine function at the other end).

2. Bringing NH₂-PEGn-N₃ into contact with an Fab fragment possessing a Q-tag and a transglutaminase, preferably for 16 hours at 37° C. This “derivatisation” step is preferably carried out with 10 times more moles of NH₂-PEGn-N₃ than moles of Fab fragment. Transglutaminase is used at a level of 15 U/μmol per Q-tag present. Desalting can optionally be carried out to remove the excess spacer not bound to the Fab fragment at the end of the step. This produces an Fab-PEGn-N₃. If the antibody or fragment has 2 Q-tags (one carried on each monomer), then the antibody or fragment can carry two PEGn-N₃

3. Synthesis of a citrullinated Cys-PEGn-peptide (i.e. a spacer coupled to a peptide at one end and having a free cysteine at another end). The DBCO is then coupled to the Cys-PEGn-citrullinated peptide at the cysteine, thus producing a DBCO-PEGn-citrullinated peptide.

4. Formation of the “click”: coupling between the N₃ and the DBCO. The DBCO-PEGn-citrullinated peptide is brought into contact with the Fab-PEGn-N₃, preferably using 10 times more moles of DBCO-PEGn-citrullinated peptide than moles of Fab-PEGn-N₃. The click reaction takes place at room temperature and is almost complete after 4 hours.

5. Producing the hybrid molecule: Fab-PEGn-N₃-DBCO-PEGn-citrullinated peptide.

Similarly, an Fab-PEGn-DBCO and an N₃-PEGn-citrullinated peptide can be obtained, then coupled to obtain Fab-PEGn-DBCO-N₃-PEGn-citrullinated peptide.

Similarly an Fab-PEGn-DBCO and an N₃-citrullinated peptide can be obtained, or an Fab-PEGn-N₃ and a citrullinated DBCO-peptide, then coupled to obtain respectively Fab-PEGn-DBCO-N₃-citrullinated peptide or Fab-PEGn-N₃-DBCO-citrullinated peptide.

The same method can be used with an antibody or another type of antibody fragment.

### Example 2: Reactivity of Purified ACPAs Against Citrullinated Peptides, with Respect to DaraFab-Cit Peptide Hybrids

The ACPAs (here ACPA, pH3 fraction) used are ACPAs obtained from the serum of patients suffering from ACPA-positive rheumatoid arthritis. Such ACPAs were obtained by a standard column affinity chromatography method known to those skilled in the art, using as bound antigens one or more of the five immunodominant peptides of the invention (see SEQ ID NO: 5, SEQ ID NO: 6, SEQ ID NO: 14, SEQ ID NO: 18 or SEQ ID NO: 19).

a. Examples with Hybrid Molecules Containing the β60-74Cit Peptide

ELISA microtitre plates were coated with 100 μl/well of a solution containing either the β60-74cit peptide (SEQ ID NO: 19), or a hybrid molecule: DaraFab-β60-74 or DaraFab-PEG-β60-74. These solutions were each used at a concentration of 5 μg/ml in PBS (Phosphate Buffered Saline) buffer and incubated overnight at 4° C. The non-citrullinated peptide and the hybrid molecules constructed with the non-citrullinated peptide (β60-74arg) were used as controls at the same concentration. The wells were then saturated in PBS 2% BSA (Bovine Serum Albumin) buffer for 1 hour at 4° C. After washing, the purified ACPAs were incubated at 1, 0.5 or 0.25 μg/ml diluted in PBS 2% BSA 2M NaCl buffer for 1 hour at 4° C. After washing, ACPA reactivity was detected using an F(ab′)2 anti-human IgG Fc secondary antibody diluted to 1/10,000 in PBS 2% BSA buffer. The results are expressed in ΔOD (change in Optical Density), corresponding to the OD obtained with the β60-74cit peptide or with the hybrid molecules constructed with the β60-74cit peptide, subtracted respectively from the OD obtained with the β60-74arg peptide or with the hybrid molecules constructed with the β60-74arg peptide.

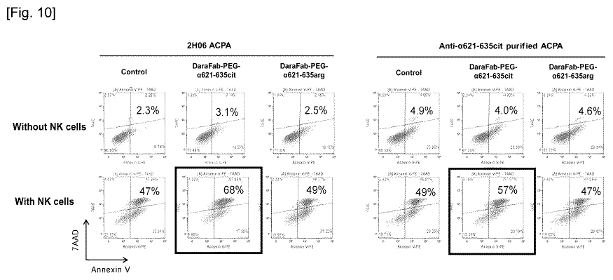

The results are shown in FIG. 3. The results show dose-dependent reactivity of the purified ACPAs against peptide β60-74 with respect to the hybrid molecules DaraFab-β60-74 and DaraFab-PEG-β60-74. The reactivity of the ACPAs with respect to the β60-74 peptide is comparatively weaker, while it exhibits an epitope density greater than that of the hybrid molecules. The epitopes are therefore more accessible on the hybrid molecules.

b. Examples with Hybrid Molecules Containing the α621-635Cit Peptide

ELISA plates were coated with 100 μl/well of a solution containing either the α621-635cit peptide (SEQ ID NO: 18), or a DaraFab-PEG-α621-635 hybrid molecule, used at a concentration of 5 μg/ml in PBS (Phosphate Buffered Saline) buffer overnight at 4° C. The non-citrullinated peptide α621-635arg and the hybrid molecules constructed with the non-citrullinated peptide (α621-635arg) were used as controls at the same concentration. The wells were then saturated in PBS 2% BSA (Bovine Serum Albumin) buffer for 1 hour at 4° C. After washing, the purified ACPAs were incubated at 4, 2 or 1 μg/ml diluted in PBS 2% BSA 2M NaCl buffer for 1 hour at 4° C. After washing, ACPA reactivity was detected using an F(ab′)2 anti-human IgG Fc secondary antibody diluted to 1/10,000 in PBS 2% BSA buffer. The results are expressed in ΔOD (change in Optical Density), corresponding to the OD obtained with the α621-635cit peptide or with the hybrid molecules constructed with the α621-635cit peptide, subtracted respectively from the OD obtained with the @621-635arg peptide or with the hybrid molecules constructed with the α621-635arg peptide.

The results are shown in FIG. 4. The results show strong dose-dependent reactivity of the purified ACPAs against α621-635Cit peptide with respect to DaraFab-PEG-α621-635cit hybrid molecules. The reactivity of the ACPAs with respect to the α621-635cit peptide is comparatively weaker, while its epitope density is greater than that of the hybrid molecules. The epitopes are therefore more accessible on the hybrid molecules.

### Example 3: Cytotoxicity of Dara-Fab Peptide (60Cit Against Cells of the Daudi Cell Line (CD38+), Induced by Purified ACPAs Against (60-74 Eluted at pH 3 (Hybrid Bridging)

The ACPAs are obtained as in Example 2.

The DaraFab-peptideCIT here represents an Fab capable of binding to CD38 bound to a PEGn spacer, itself coupled to an azide that is covalently bound to a cyclooctyne (DBCO) that is coupled to a peptide of SEQ ID NO: 19. DaraFab-peptideNCIT here represents an Fab capable of binding to CD38 bound to a PEGn spacer, itself coupled to an azide that is covalently bound to a cyclooctyne (DBCO) that is coupled to a peptide of SEQ ID NO: 19, in which the arginyl residues have not been substituted by citrullyl residues (i.e. the peptide is not citrullinated). n represents a number between 1 and 10, preferably 1, 2, 3, 4 or 8.

Cells from the Daudi cell line (CD38+) were deposited in 96-well plates, in an amount of 50,000 cells/well in complete RPMI medium (10% FBS, 1% glutamine, 1% penicillin/streptomycin) and incubated for 1 hour at 37° C. with 10 μg/ml of DaraFab-peptideCIT or DaraFab-peptideNCIT hybrids. After washing, human IgG (negative control) or purified anti-β60-74 ACPAs eluted at pH 3, at 5 μg/ml for 20 hours at 37° C. were added, before labelling with Annexin V (apoptosis marker). The cells were then collected and analysed by flow cytometry (BD Biosciences, CANTO II). The values shown in the first 3 columns correspond to the percentage of Annexin V-positive cells. Δ(CIT-NCIT) (%) represents the difference between the percentages of Annexin V-positive cells in the presence of DaraFab-peptideCIT versus NCIT, divided by the percentage of Annexin V-positive cells in the presence of DaraFab-β60-74peptideNCIT. This indicator shows the modulation of apoptosis in the presence of IgG or ACPA.

The results are presented in Table 2.

TABLE 2

| | | DaraFab- | DaraFab- | Δ(CIT − NCIT) |
|---|---|---|---|---|
| Antibody | Untreated | peptideCIT | peptideNCIT | (%) |
| IgG | 21.9 | 21.4 | 22.6 | −5% |
| | 24 | 24.6 | 23.4 | +5% |
| | 9.57 | 9.9 | 9.45 | +4% |
| | 26.2 | 25.1 | 27.3 | −8% |
| ACPA | 28.9 | 32.6 | 28.5 | +14% |
| | 26.4 | 29 | 25.8 | +12% |
| | 26.8 | 31.6 | 26.1 | +21% |
| | 27.9 | 32.4 | 27.1 | +19% |

Table 2 represents the quantification of the apoptosis of Daudi line cells (CD38+) coated with DaraFab-β60-74CIT, after braidging by the purified ACPAs eluted at pH3. Synthesis of four independent experiments.

The results show an increase in cell death in the presence of ACPAs when the DAUDI cells were treated with the DaraFab-β60-74CIT hybrid compared to those treated with DaraFab-β60-74NCIT. The increase in cell death in the presence of the citrullinated hybrid molecule (DaraFab-β60-74CIT) compared to the non-citrullinated form (DaraFab-β60-74NCIT) after bridging by ACPAs is on average 16.5%.

Cells from the Daudi cell line (CD38+) were deposited in 96-well plates, in an amount of 50,000 cells/well in complete RPMI medium (10% FBS, 1% glutamine, 1% penicillin/streptomycin) and incubated for 1 hour at 37° C. with 10 μg/ml of DaraFab-β60-74CIT or DaraFab-β60-74NCIT hybrids. After washing, human IgG (negative control) or purified anti-β60-74 ACPAs eluted at pH 3 at 5 μg/ml were added and incubated for 20 hours at 37° C. A cell viability test (MTT: colorimetric marker of cellular mitochondrial activity) was carried out. The cells were then removed and the OD analysed by spectrometry (Thermofisher Multiskan Fc). The values represented correspond to the ODs obtained under each experimental condition.

Δ(CIT-NCIT) (%) represents the difference between the OD obtained in the presence of DaraFab-β60-74CIT versus NCIT, divided by the OD obtained in the presence of DaraFab-β60-74NCIT.

The results are presented in Table 3.

TABLE 3

| | | DaraFab- | DaraFab- | Δ(CIT − NCIT) |
|---|---|---|---|---|
| Antibody | Untreated | peptideCIT | peptideNCIT | (%) |
| IgG | 0.716 | 0.717 | 0.696 | −3% |
| | 0.886 | 0.825 | 0.792 | −4% |
| | 1.055 | 1.018 | 0.996 | −2% |
| ACPA | 0.571 | 0.497 | 0.589 | −16% |
| | 0.61 | 0.566 | 0.721 | −21% |
| | 0.822 | 0.702 | 0.829 | −15% |

Table 3 shows the evaluation of cell viability of the Daudi line cells (CD38+) coated with DaraFab-β6074Cit after bridging by the purified ACPAs eluted at pH 3. Syntheses of three independent experiments.

The results show a decrease in cell viability in the presence of ACPAs when the Daudi cells were treated with the DaraFab-β60-74CIT hybrid compared to those treated with DaraFab-β60-74NCIT.

The decrease in cell viability in the presence of the citrullinated hybrid molecule (DaraFab-β60-74CIT) compared to the non-citrullinated form (DaraFab-860-74NCIT) after bridging by ACPAs is on average 17.33%.

### Example 4: Cytotoxicity of DaraFab-PEG-β60-74Cit Against the BAEV B Cell Line (BC-9) Produced from the Lymphocytes of a Patient with Rheumatoid Arthritis, after Bridging by Purified Anti-660-74 ACPAs

The ACPAs are obtained as in Example 2.

Cells of the BC-9 cell line (B BAEV) from the blood lymphocytes of a patient with rheumatoid arthritis were deposited in 96-well plates, in an amount of 100,000 cells/well in complete IMDM medium (10% FBS, 1% glutamine, 1% penicillin/streptomycin) and incubated for 1 hour at 37° C. with 10 μg/ml of DaraFab-PEG-β60-74cit and DaraFab-PEG-β60-74arg hybrids. After washing, purified anti-β60-74 ACPAs eluted at pH 3, at 5 μg/ml for 20 hours at 37° C., were added before labelling with 7-AAD (late apoptosis marker). The cells were then removed and analysed by flow cytometry (BD Biosciences, CANTO II). The data presented corresponds to the means of four independent experiments. Statistical analysis was performed using the Student's t-test (*p<0.05).

DaraFab-PEG-β60-74cit here represents an Fab capable of binding to CD38 bound to a

PEGn spacer, itself coupled to an azide that is covalently bound to a cyclooctyne (DBCO) that is coupled to a peptide of SEQ ID NO: 19. DaraFab-PEG-β60-74arg here represents an Fab capable of binding to CD38 bound to a PEGn spacer, itself coupled to an azide that is covalently bound to a cyclooctyne (DBCO) that is coupled to a peptide of SEQ ID NO: 19, in which the arginyl residues have not been substituted by citrullyl residues (i.e. the peptide is not citrullinated). n represents a number between 1 and 10, preferably 1, 2, 3, 4 or 8. The results (presented in FIG. 5) thus show that there is a significant reduction in cell viability (left-hand part) in the presence of the purified ACPAs when the BC-9 cells have previously been incubated with the citrullinated hybrid DaraFab-PEG-β60-74cit compared to the viability of those incubated with DaraFab-PEG-β60-74arg. At the same time, there is a significant increase in late apoptosis (right-hand part) under these same conditions.

### Example 5: Cytotoxicity of the Dara-Fab Peptide-660Cit and Dara-Fab Peptide-α621Cit Hybrids Against a Differentiated BAEV B Cell Line Derived from Lymphocytes of a Patient with Rheumatoid Arthritis), after Bridging of the Hybrids by Purified Anti-660-74 ACPAs Eluted at pH 3

The ACPAs are obtained as in Example 2.

Cells from the BC-9 cell line (B BAEV) were differentiated (for 7 days) in order to increase expression of the CD38 marker on their surface as shown in FIG. 6.

DaraFab-PEG-α621-635 here represents an Fab fragment capable of binding to CD38 linked to a PEGn spacer, itself coupled to an azide that is covalently bound to a cyclooctyne (DBCO) that is coupled to a PEGn spacer that is itself bound to a peptide of SEQ ID NO: 18. DaraFab-PEG-α621-635cit represents the hybrid in which the peptide is citrullinated and DaraFab-PEG-α621-635arg represents the case in which the peptide is not citrullinated. n represents a number between 1 and 10, preferably 1, 2, 3, 4 or 8.

DaraFab-PEG-β60-74 here represents an Fab capable of binding to CD38 linked to a PEGn spacer, itself coupled to an azide that is covalently bound to a cyclooctyne (DBCO) that is coupled to a PEGn spacer that is itself bound to a peptide of SEQ ID NO: 19. DaraFab-PEG-β60-74cit represents the hybrid in which the peptide is citrullinated and DaraFab-PEG-β60-74arg represents the case in which the peptide is not citrullinated. n represents a number between 1 and 10, preferably 1, 2, 3, 4 or 8.

Cells from the BC-9 cell line (B BAEV) from the blood lymphocytes of a patient with rheumatoid arthritis subjected for 7 days to a plasma cell differentiation protocol were deposited in 96-well plates, in an amount of 100,000 cells/well in complete IMDM medium (10% FBS, 1% glutamine, 1% penicillin/streptomycin), then incubated for 30 minutes at 37° C. with 10 μg/ml of DaraFab-PEG-β60-74cit/DaraFab-PEG-β60-74arg and DaraFab-PEG-α621-635cit/DaraFab-PEG-α621-635arg hybrids. After washing, human IgG (negative control) or purified anti-β60-74 ACPAs eluted at pH 3, at 5 μg/ml for 24 or 48 hours at 37° C. were added, before labelling with Annexin V (early apoptosis marker) and 7-AAD (late apoptosis marker). The cells were then collected and analysed by a cell death analyser (Luminex, MUSE). The values shown correspond to the percentage of Annexin-positive cells. Δ(cit-arg) (%) represents the difference between the percentage of Annexin V-positive cells in the presence of DaraFab-PEG-β60-74cit or DaraFab-PEG-α621-635cit and the percentage of Annexin V-positive cells in the presence of DaraFab-PEG-β60-74arg or DaraFab-PEG-α621-635arg, divided by the percentage of Annexin V-positive cells in the presence of DaraFab-PEG-β60-74arg or DaraFab-PEG-α621-635arg.

The results are presented in Table 4.

TABLE 4

| | 24 hours | | | | 48 hours | | | |
|---|---|---|---|---|---|---|---|---|
| | DaraFab- | | DaraFab- | | DaraFab- | | DaraFab- | |
| | PEG- | | PEG- | | PEG- | | PEG- | |
| | α621-635 | | β60-74 | | α621-635 | | β60-74 | |
| | cit | | arg | | cit | | arg | |
| +IgG | 19% | 18% | 18% | 17% | 27% | 24% | 26% | 25% |
| +ACPA | 28% | 22% | 23% | 17% | 41% | 29% | 33% | 24% |
| pH 3 | | | | | | | | |

| | | | | |
|---|---|---|---|---|
| Δcit − | +30% | +32% | +40% | +41% |
| arg | | | | |

Table 4 represents the quantification of apoptosis

The results indicate the quantification of apoptosis induced in the presence of ACPAs when BC-9 cells have been pre-treated with DaraFab-PEG-β60-74cit or DaraFab-PEG-α621-635cit, compared to those pre-treated with the non-citrullinated forms DaraFab-PEG-β60-74arg and DaraFab-PEG-α621-635arg.

The increase in apoptosis in the presence of citrullinated hybrid molecules (DaraFab-β60-74cit and DaraFab-PEG-α621cit) compared to non-citrullinated forms, after bridging by purified ACPAs, is around 30% after 24 hours and 40% after 48 hours.

### Example 6: Interaction of the DaraFab-PEG-α621-635 Hybrid and Purified Anti-α621-635Cit ACPAs or 2H06 ACPA on the Surface of the KMS-12-PE Cell Line Derived from Human Myeloma or Malignant Plasmacytoma

The ACPAs (here, purified ACPAs) are ACPAs obtained from the serum of ACPA-positive rheumatoid arthritis patients. Such ACPAs were obtained by a standard column affinity chromatography method known to those skilled in the art, using as bound antigens one or more of the five immunodominant peptides of the invention (see SEQ ID NO: 5, SEQ ID NO: 6, SEQ ID NO: 14, SEQ ID NO: 18 or SEQ ID NO: 19).

The ACPA also used here is a recombinant monoclonal human ACPA (clone 022014CCP14CFCT2H06, known as 2H06). Such an ACPA can be obtained as described in Titcombe P. J., Wigerblad G., Sippl N., Zhang N., Shmagel A. K., Sahlstrom P., Zhang Y., Barsness L. O., Ghodke-Puranik Y., Baharpoor A., et al. Pathogenic Citrulline-Multispecific B Cell Receptor Clades in Rheumatoid Arthritis. Arthritis & Rheumatology 2018, 70 (12), 1933-1945. https://doi.org/10.1002/art.40590. The 2H06 ACPA VH has Genbank accession number MH629710.1, and VL has accession number MH629700.1.

DaraFab-PEG-α621-635 Cit/Arg hybrids were labelled with AlexaFluor®647 fluorochromes and purified anti-α621-635cit ACPAs as well as 2H06 ACPA were labelled with AlexaFluor®488 fluorochromes using commercial kits (Lightning Link, Abcam). Cells from the KMS-12-PE human myeloma cell line were deposited in a 96-well plate in an amount of 200,000 cells/well in complete RPMI medium (10% FCS, 1% glutamine, 1% penicillin/streptomycin) and incubated for 30 minutes at 4° C. with 10 μg/ml of DaraFab-PEG-α621-635 Cit/Arg hybrids labelled with AlexaFluor®647, then incubated with purified anti-α621-635cit ACPAs or with 2H06 ACPA labelled with AlexaFluor®488 for 30 minutes at 4° C. at 5 μg/ml. After washing in a 2% PBS BSA buffer, the KMS-12-PE cells were collected and analysed by flow cytometry (BD Biosciences, Canto II).

The results presented in FIG. 7 show interaction of the DaraFab-PEG-α621-635 Cit hybrid molecules and anti-α621-635cit ACPAs or 2H06 ACPA on the surface of the KMS-12-PE myeloma cell line, visualised by the appearance of a double-positive AF647/AF488 population. This interaction is specific and associated with the peptide because it is not observed in the presence of the DaraFab-PEG-α621-635 Arg hybrids.

### Example 7: Phagocytosis of the Differentiated KMS-12-PE Myeloma Cell Line by Macrophages in the Presence of DaraFab-PEG-α621-635 and 2H06 ACPA

The 2H06 ACPA is obtained as in Example 6.

(A) Cells from the KMS-12-PE myeloma cell line were differentiated for 4 days to increase expression of the CD38 marker on their surface, using a mixture of cytokines consisting of IFN-α at 100 ng/ml, IL-6 at 50 ng/ml, and IL-15 at 10 ng/ml. The cells were then labelled with CFSE (carboxyfluorescein succinimidyl ester, fluorescent intracellular marker) and placed in the presence of Daratumumab (anti-CD38 monoclonal AC used as a positive control) or in the presence of DaraFab-PEG-621-635 Cit/Arg hybrids for 30 minutes at 10 μg/ml in complete RPMI medium at 37° C. The cells were then incubated with 2H06 ACPA at 50 μg/ml for 30 minutes in complete RPMI medium at 37° C. and brought into contact, without washing, with human macrophages (differentiated in vitro in the presence of M-CSF (100 ng/ml) from CD14+ monocytes isolated from the peripheral blood of a healthy subject, unlabelled), in an amount of 1 KMS-12-PE cell per 1 macrophage, for 2 hours at 37° C. The macrophages were then separated and analysed by flow cytometry using Cd11b BV421 labelling (macrophage-specific membrane marker). The different cell populations are expressed as a percentage of the total cell population and include the percentage of CFSE/BV421 double-positives (macrophages that have phagocytosed KMS-12-PE cells) and the percentage of residual KMS-12-PE target population. (B) The data are representative of four independent identical experiments. Statistical analysis was performed using the paired Student's t-test (** p<0.01; *** p<0.001).

(A) The results presented in FIG. 8 show that phagocytosis activity is enhanced in the presence of hybrids DaraFab-PEG-α625-631 Cit and 2H06 ACPA. A 70% increase in double-positive events is observed and associated with a 73% decrease in the target population. In addition, the phagocytosis activity obtained in the presence of the DaraFab-PEG-α625-631 Cit hybrids and 2H06 ACPA is higher than that obtained with the Daratumumab control. (B) This experiment was performed four times and a specific and significant increase (*** p<0.001, paired t-test) of 78% of the double-positive population was observed in the presence of the hybrids DaraFab-PEG-α625-631 Cit and 2H06 ACPA, as well as a significant reduction (** p<0.01, paired t-test) of 75% of the target population.

### Example 8: Phagocytosis of the Differentiated Myeloma KMS-12-PE Cell Line by Macrophages in the Presence of Purified DaraFab-PEG-β60-74 or DaraFab-PEG-α621-635 and ACPAs, Respectively Anti-60-74Cit or Anti-α621-635Cit

The 2H06 ACPA is obtained as in Example 6.

Cells from the KMS-12-PE myeloma cell line were differentiated for 4 days in order to increase expression of the CD38 marker on their surface, using a mixture of cytokines consisting of IFN-α at 100 ng/ml, IL-6 at 50 ng/ml, and IL-15 at 10 ng/ml. The cells were then labelled with CFSE (carboxyfluorescein succinimidyl ester, fluorescent intracellular marker) and placed in the presence of Daratumumab (anti-CD38 monoclonal antibody used as a positive control) or in the presence of DaraFab-PEG-α621-635 Cit/Arg hybrids or DaraFab-α621-635 Cit/Arg hybrids or DaraFab-PEG-β60-74 Cit/Arg or DaraFab-β60-74 Cit/Arg hybrids, for 30 minutes at 10 μg/ml in complete RPMI medium at 37° C. The cells were then incubated with the purified anti-α621-635cit or anti-ß60-74cit

ACPAs at 5 μg/ml for 30 min in complete RPMI medium at 37° C. The KMS-12-PE cells were brought into contact, without washing, with human macrophages, differentiated in vitro in the presence of M-CSF (100 ng/ml), from CD14+ monocytes isolated from the peripheral blood of a healthy subject, unlabelled, in an amount of 1 KMS-12-PE cell per 1 macrophage, for 2 hours at 37° C. The macrophages were then separated and subsequently analysed by flow cytometry using Cd11b

BV421 labelling (macrophage-specific membrane marker).

The results are presented in FIG. 9 and show that phagocytosis activity increases in the presence of DaraFab-peptide α625-631 Cit or DaraFab-peptide β60-74 Cit hybrids and the corresponding purified ACPAs, anti-α621-635cit or anti-β60-74cit. In addition, the phagocytosis activity obtained in the presence of the DaraFab-peptide α625-631 Cit hybrids and the purified anti-α625-631cit ACPAs is higher than that obtained with the Daratumumab control. Under the conditions using the DarFabβ60-74cit hybrids and the purified anti-β60-74cit ACPAs, the phagocytosis activity is equivalent to that obtained with the Daratumumab control.

### Example 9: Induction of Apoptosis in Cells of the Differentiated KMS-12-PE Myeloma Cell Line by NK Cells in the Presence of DaraFab-PEG-α621-635 and Purified Anti-α621-635Cit ACPA or 2H06 ACPA

The purified ACPAs and the 2H06 monoclonal antibody are obtained as described in Example 6.

Cells from the KMS-12-PE myeloma cell line were differentiated for 3 days in order to increase expression of the CD38 marker on their surface, using a mixture of cytokines comprising IFN-α at 100 ng/ml, IL-6 at 50 ng/ml, and IL-15 at 10 ng/ml. In a 96-well plate, 100,000 cells/well were incubated with the DaraFab-α621-635 Cit hybrid molecules for 30 min in complete RPMI medium at 37° C. at 10 μg/ml then with 2H06 ACPA at 50 μg/ml or with the purified anti-α621-635cit ACPAs at 10 μg/ml, in the presence of freshly purified NK cells (labelled with Cell Tracker Violet) used as effector cells, also at 100,000 cells/well, for 16 hours at 37° C. The DaraFab-α621-635 Arg hybrid molecules, used as specificity controls, were incubated under the same conditions. As negative controls, the KMS-12-PE cells were incubated in the absence of hybrids, alone or in the presence of NK cells. Cell lysis was measured by flow cytometry using labelling with the fluorescent probes 7-AAD and Annexin V-PE.

The results are presented in FIG. 10 and show that the DaraFab-PEG-α621-635 Cit hybrids, in the presence of purified anti-α621-635cit ACPAs or 2H06 ACPAs, are capable of specifically increasing lysis by ADCC caused by NK cells of the KMS-12-PE target cells. The destruction of target cells increases from 47% to 68% in the presence of 2H06 ACPA and from 49% to 57% in the presence of purified anti-α621-635cit ACPAs.

1. A hybrid molecule comprising at least one fibrin-derived peptide having at least one citrullyl residue, said peptide being covalently bound to at least one antibody or to at least one antibody fragment, said antibody or fragment being capable of binding to CD38 and/or CD138, one or more spacers being optionally present between said peptide and said antibody or said fragment.

2. A hybrid molecule according to claim 1, wherein said peptide is covalently bound to a spacer, said spacer being itself covalently bound to said antibody or antibody fragment.

3. A hybrid molecule according to either one of the preceding claims, wherein said peptide is derived from all or part of the sequence of the α or β chain of a vertebrate fibrin by substitution of at least one arginyl residue by a citrullyl residue, said vertebrate fibrin preferably being a mammalian, preferably a human, fibrin.

4. A hybrid molecule according to any one of the preceding claims, wherein said spacer is a polymer containing one or more repeat units containing the ether group, said spacer preferably being polyethylene glycol of formula PEGn, in which n represents an integer between 1 and 100, preferably between 1 and 10 and in particular 1, 2, 3, 4 or 8.

5. A hybrid molecule according to any one of the preceding claims, wherein said peptide comprises at least one citrullyl residue, and is selected from the group consisting of:
a) a peptide defined by the sequence X1PAPPPISGGGYX2AX3 (SEQ ID NO: 1) in which X1, X2, and X3 each represent a citrullyl residue or an arginyl residue, and at least one of the X1 or X2 or X3 residues is a citrullyl residue:
b) a peptide defined by the sequence GPXIVVEX2HQSACKDS (SEQ ID NO: 2) in which X1 and X2 each represent a citrullyl residue or an arginyl residue, and at least one of the X1 or X2 residues is a citrullyl residue:
c) a peptide defined by the sequence SGIGTLDGFX1HX2HPD (SEQ ID NO:3) in which X1 and X2 each represent a citrullyl residue or an arginyl residue, and at least one of the X1 or X2 residues is a citrullyl residue:
d) a peptide defined by the sequence VDIDIKIX1SCX2GSCS (SEQ ID NO:4) in which X1 and X2 each represent a citrullyl residue or an arginyl residue, and at least one of the X1 or X2 residues is a citrullyl residue:
e) a peptide defined by the sequence X1GHAKSX2PVX3GIHTS (SEQ ID NO: 12) in which X1, X2 and X3 each represent a citrullyl residue or an arginyl residue, and at least one of the X1 or X2 or X3 residues is a citrullyl residue:
f) a peptide comprising at least 5 consecutive amino acids, including at least one citrullyl residue, from one of peptides a) to e) above.

6. A hybrid molecule according to any one of claims 1-4, wherein said peptide is selected from the group consisting of: SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23, more particularly selected from the group consisting of: SEQ ID NO: 5, SEQ ID NO: 6, SEQ ID NO: 14, SEQ ID NO: 18 and SEQ ID NO: 19, more particularly SEQ ID NO: 18 and SEQ ID NO: 19.

7. A hybrid molecule according to any one of the preceding claims, wherein the fragment is selected from fragments, scFc, Fv, Fab or F(ab′)2.

8. A hybrid molecule according to any one of the preceding claims, wherein said antibody or F(ab′)2 fragment is a bispecific antibody or F(ab′)2 fragment, directed against:
CD38 and another plasma cell target, or
CD138 and another plasma cell target, or
CD38 and CD138.

9. A hybrid molecule according to any one of the preceding claims, said molecule being represented by:
an antibody fragment capable of binding to CD38 bound to a PEGn spacer, itself coupled to an azide covalently bound to a cyclooctyne coupled to a peptide represented by SEQ ID NO: 19, or SEQ ID NO: 18,
an antibody fragment capable of binding to CD38 bound to a PEGn spacer, itself coupled to an azide covalently bound to a cyclooctyne coupled to a PEGn spacer, itself bound to a peptide represented by SEQ ID NO: 19 or SEQ ID NO: 18,
n preferably representing an integer between 1 and 10, more particularly 1, 2, 3, 4 or 8.

10. A hybrid molecule according to any one of the preceding claims for use as a medicinal product, in particular for use in the treatment of autoimmune diseases associated with the production of anti-citrullinated protein autoantibodies, in particular Gougerot-Sjögren syndrome and rheumatoid arthritis.

11. A hybrid molecule according to any one of the preceding claims, for use as a medicinal product, in combination with a second hybrid molecule, said second molecule comprising at least one Fc antibody fragment covalently bound to at least one fibrin-derived peptide carrier of a citrullyl residue, a spacer being optionally present between said Fc fragment and said peptide.
